(19) United States
(12) Patent Application Publication
(10) Pub. No.: US 2006/0121502 A1
(43) Pub. Date: Jun. 8, 2006

(54) Microfluidics apparatus for cantilevers and methods of use therefor

(76) Inventors: Robert Cain, Rockville, MD (US); Paul Mirer, Rockville, MD (US); Salvatore Seminara, Chicago, IL (US); Timothy Seeley, Bethesda, MD (US); Sebastian Kossek, Phoenix, AZ (US); Thomas Wiggins, Columbia, MD (US)

Correspondence Address: LAWSON & WEITZEN, LLP, 88 BLACK FALCON AVE, SUITE 345, BOSTON, MA, 02210, US

(21) Appl. No.: 11/254,348

(22) Filed: Oct. 20, 2005

Related U.S. Application Data
(63) Continuation-in-part of application No. 10/992,368, filed on Nov. 18, 2004.
Application No. 10/992,368 is a division of application No. 10/054,760, filed on Nov. 13, 2001, now Pat. No. 6,926,864.
Application No. 10/054,760 is a continuation-in-part of application No. 10/036,733, filed on Nov. 9, 2001, now abandoned.
(63) Continuation-in-part of application No. 10/705,434, filed on Nov. 10, 2003.

(51) Int. Cl. C12Q 1/68; G01N 33/53; G06F 19/00; C12M 1/34

(52) U.S. Cl. 435/6; 435/7.1; 435/287.2; 702/20

(57) ABSTRACT

A microfluidics device includes a plurality of interaction cells and fluid control means including i) means for providing to the interaction cells a preparation fluid, ii) means for providing to the interaction cells a sample fluid, wherein each interaction cell receives a different sample fluid, and iii) means for thermal control. A plurality of cantilevers may be disposed in each of the interaction cells, the cells or chambers formed by a cartridge bottom and top that form the device, wherein each of the plurality of cantilevers is configured to deflect in response to an interaction involving a component of the sample fluid. The cantilevers in each cell are attached to a reference plane that controls for environmental factors or non-analyte deflections.

The present application is a continuation-in-part of the application entitled “Microfluidics Apparatus and Methods of Use Therefor,” Ser. No. 10/992,368 filed Nov. 18, 2004, which is a divisional of the application Ser. No. 10/054,760 filed Nov. 13, 2001, now U.S. Pat. No. 6,930,168 B2 issued Aug. 16, 2005, which is a continuation-in-part of the application filed Nov. 9, 2001, Ser. No. 10/036,733, and this application is a continuation-in-part of the application entitled “Multiplex Illuminator and Device Reader for Microcantilever Array”, Ser. No. 10/705,434, filed Nov. 10, 2003, the contents of each of which are incorporated herein by reference in their entireties.

### TECHNICAL FIELD

The present invention relates to cantilever apparatus for chemical analysis, and in particular to methods and apparatuses for performing chemical analysis of biomaterials with a microfluidics device using microcantilevers with a control for reference plane detection of deflection.

### BACKGROUND OF THE INVENTION

It is known that thin bimorph microcantilevers can undergo bending (deflection) due to differential stresses following exposure to and binding of a compound from their environment, for example in a fluid sample.

Microcantilevers having spring constants less than 0.1 N/m are sensitive to stress differentials that arise as a result of interactions between extremely small amounts of a substrate material on a surface of the microcantilever and one or more materials in a sample. For a given microcantilever with a specially designed coating layer, the deflection yields information about components of the environment to which the microcantilever is exposed.

Microcantilevers are capable of detecting calorimetric enzyme-mediated catalytic biological reactions with femtoJoule resolution. (Thundat et al., “Microcantilever Sensors”, Microscale Thermophysical Engr. 1, pgs. 185-199, 1997.) Further, oligonucleotide interactions within a sample can be detected using a monolithic array of test sites formed on a surface to which the sample is applied as shown in U.S. Pat. No. 5,653,939.

It is also known to provide integrated chips to categorize molecules in a biochemical sample. For example, U.S. Pat. No. 6,123,819 to Peeters discloses a design for an integrated chip having an array of electrodes at the atomic or nano scale. The chip can be used to characterize single molecules in a solution such as individual proteins, complex protein mixtures, DNA, or other molecules.

In recent years, microfluidics technology employing microcantilevers has emerged to provide a “lab-on-a-chip” for chemical analysis of biomaterials. For example, U.S. Pat. No. 6,054,277 to Furcht et al. discloses a genetic testing system that includes an integrated, unitary microchip-based detection device with microfluidic controls. The device employs a microcantilever sensor to detect a biochemical reaction in a single detection chamber having capillary interconnects. However, to analyze a number of solutions simultaneously, it would be necessary to utilize an equal number of these chips. In practice of methods involving multiple sets of cantilevers for analyzing a number of sample solutions, environmental factors are found to vary over time or from sample-to-sample or device-to-device. Such factors include thermal variation within an opto-mechanical assembly associated with a chip; “drift” or variation in electronics, such as temporal deviations in current; and differences in refractive index from sample to sample. There is a need for methods and devices to standardize and control for variation due to these and other factors in cantilever sensors.

### SUMMARY OF THE INVENTION

The invention in one aspect provides a cantilever sensor device comprising cantilevers (also known as cantilever fingers), the device comprising a plurality of cantilevers, each of the cantilevers having a base end attached to a base and a free end that deflects as a response to a presence of an analyte in a sample in an environment, the base further comprising a reference plane as a control for “non-cantilever signals” in the environment. The phrase, “non-cantilever signals” as used herein and in the claims means a class of signals that are not related to deflection or bending of the cantilevers in response to the presence of an analyte, i.e., can also be expressed as “non-analyte” signals, so that the reference planes measures responses that are not related to the basis of deflection for which the cantilevers are configured. In addition to the embodiment of cantilevers deflecting in response to the presence of an analyte, the cantilevers in another embodiment deflect in response to a change in configuration of a ligand attached to a surface of the cantilever.

The device in one embodiment is arranged such that the reference plane is co-contiguous to the base, and the base, reference plain and cantilever fingers are substantially co-planar or occupy parallel planes. In certain cantilevers, a material is deposited on a base to form the cantilevers, so that the cantilever fingers and base may not be co-planar, however the planes of each of the cantilevers and the base are parallel, i.e., the planes of the cantilevers and the base having the reference plane are parallel or are substantially parallel.

In general a cantilever is manufactured to have a “device layer”, comprising, for example, single crystal silicon, and a “carrier layer”, comprising silicon oxide, to make up a “silicon on insulator” wafer or chip or die. The cantilevers are cut out of the device layer, which is exemplified by a single crystal silicon, and removal of the carrier layer releases the cantilevers while the base with the reference plane remains made up of the carrier layer and device layer. The layers together may also be said to comprise a “substrate” or support surface. Accordingly, in embodiments of the device the cantilevers, the base and the reference plane comprise a die having layers of substantially the same substrate materials, prior to release of the cantilevers by removal of the carrier layer. Thus the cantilevers are built into a die or wafer, and are released by further milling of the die.

A surface of the cantilever in certain embodiments further includes a coating comprising a ligand for the analyte. A ligand is alternatively defined as a “capture molecule”. In this embodiment, the cantilever deflects following addition of a sample containing the analyte, because the analyte binds to the ligand or capture molecule on the surface.

In an alternative embodiment, the surface includes a coating however this coating has not been configured as a ligand for an analyte. In the latter alternative embodiment, the cantilever is configured to measure a change in configuration of an analyte present on the surface of the cantilever, so that deflection occurs due to a change in configuration rather than due to binding of a ligand. In general, a first side of the cantilevers, the base and the reference plane each comprises the coating, and a second side of the cantilever lacks this coating.

It is not necessary that the cantilevers and base comprise the same materials, for example, the cantilevers are exemplified by silicon nitride or plastic deposited on a silicon base. Further, the coatings need not be the same. For example, the cantilevers are exemplified by having a coating that is a metal layer, a linker molecule or a capture molecule, and the base and reference plane either include the metal layer, a linker molecule or a capture molecule, or lack the metal layer, a linker molecule or a capture molecule. In general, the coating on the cantilevers is a metal layer which is reflective, and the reference plane is similarly coated with the reflective metal layer.

The reference plane measures non-analyte signals comprising at least one from the group: thermal variation within the opto-mechanical assembly; refractive index difference in sample fluids; and drift in electronics, i.e., the reference plane is used to determine the extent of any signals not related to the cantilever movement, or due to factors not related to the analytes for which cantilevers are configured to respond. The device drift in electronics is a variation in at least one of: in intensity of a laser source, movement of a laser source, sensitivity of an optical detector, such as position sensitive detector (PSD) or charge couple device (CCD), processing of signal from the optical detector.

In general, the device further includes an algorithm embedded in a computer-readable medium, so that the algorithm integrates a sum of non-analyte signal data and subtracts that sum from an amount of cantilever responses. The cantilever responses which are measured include at least one change of: extent of deflection; resonance frequency; higher flexural mode; phase angle of the flexural mode with respect to an actuation mechanism; and a quality factor of the flexural modes.

Another embodiment of the invention herein is a cantilever platform which has a plurality of interaction cells, each interaction cell comprising an inlet for receiving a sample fluid; and at least one test cantilever having a movable end and a fixed base disposed in each interaction cell, and at least one reference plane fixed relative to the cantilever base, such that the test cantilever and the reference plane comprise a die having layers of substantially the same substrate materials.

The cantilever further includes, in each interaction cell, at least one outlet whereby fluid may flow out of the cell. The cantilever platform can further include a housing, which is related embodiments is a fluidics cartridge having the plurality of interaction cells, inlets and outlets, and a lid. Further, the die having the test cantilevers and reference plane disposed in the interaction cell is removable.

The cantilever platform generally includes in each interaction cell a plurality of test cantilevers, although a single cantilever is within the scope of the embodiments herein. The plurality of test cantilevers is contiguous to the reference plane, i.e., is adjacent to the reference plane and co-planar, such that the test cantilever or cantilevers all of the lie in substantially the same plane as the reference plane, or all lie in a plane that is parallel to the reference plane.

In general, the fluidics cartridge and the fluid entering the cells are temperature controlled. Further, the housing is removably inserted into an illuminator-reader apparatus for measuring a movement of a test cantilever and an apparent movement of the reference plane. The illuminator-reader apparatus further comprises means for generating and focusing a plurality of laser beams, wherein at least one of the plurality of laser beams focuses respectively on each of the test cantilevers and on the reference plane.

Another embodiment of the invention provided herein is a method for analyzing in each of a plurality of sample fluids a quantity of an analyte or a change in conformation of a ligand in which non-analyte factors are detected, the method comprising: contacting at least one of a plurality of interaction cells with at least one the sample fluids, wherein each of the interaction cells comprises at least one test cantilever and at least one reference plane; detecting in each interaction cell a response of the test cantilever and an amount of an apparent deflection of the reference plane; and calculating a difference in the response of the test cantilever and the amount of the apparent deflection of the reference plane cantilever due to non-analyte factors, wherein the quantity of the analyte or change in conformation of the ligand is calculated.

In certain embodiments, the test cantilever is configured to deflect in response to the presence of a ligand selected from a group consisting of a protein and a nucleic add. For example, the nucleic acid is RNA. Alternatively, the nucleic acid is DNA. Further, the protein is selected from an epitope, an enzyme, and a synthetic polypeptide. In certain embodiments, the ligand or the analyte is a substrate for an enzyme.

The phrase, “substrate for an enzyme” as used in the context of a ligand or analyte herein, means a molecule capable of being chemically changed by digestion with an enzyme. This usage is distinct from the term, “substrate” as used in the context of a die for a cantilever chip or wafer, in which context the term means a planar layer of a material used to manufacture that chip or wafer.

In alternative embodiments, the ligand or the analyte is a hormone. The hormone is selected from the group consisting of a steroid and a polypeptide. In alternative embodiments, the ligand or analyte is selected from the group consisting of an antibody and an antigen.

In alternative embodiments, the method further includes mounting the interaction cells in a housing comprising means for controlling temperature of the cells and means for controlling temperature of the fluid entering the cells. Detecting an amount of an apparent deflection is measuring a sum of non-analyte signals. The non-analyte factors comprise at least one of: thermal variation within the opto-mechanical assembly; refractive index difference in sample fluids; and drift in electronics. In certain embodiments, calculating the difference further comprises using a computer algorithm embedded in a computer readable medium.

Yet another embodiment of the invention provides a microfluidics device having: four interaction cells, each interaction cell being configured to contain at least four test cantilevers and one reference plane; and fluid control means for providing to the interaction cells a sample fluid, wherein each interaction cell receives a different sample fluid. In a particular embodiment, the device thus contains 16 cantilevers and four reference planes among the four cells.

### BRIEF DESCRIPTION OF THE DRAWINGS

The foregoing features of the invention will be more readily understood by reference to the following detailed description, taken with reference to the accompanying drawings, in which:

FIG. 1 is a graphical illustration showing a bottom view of an apparatus for performing microfluidic analysis in accordance with an embodiment of the invention;

FIG. 2 is a graphical illustration showing a top view of the apparatus of FIG. 1;

FIG. 3 is a graphical illustration of the embodiment of FIG. 1 showing all valves closed;

FIG. 4 is a graphical illustration of the embodiment of FIG. 1 showing a linker solution added to a first interaction cell;

FIG. 5 is a graphical illustration of the embodiment of FIG. 1 showing a linker solution added to a second interaction cell;

FIG. 6 is a graphical illustration of the embodiment of FIG. 1 showing a linker solution added to a third interaction cell;

FIG. 7 is a graphical illustration of the embodiment of FIG. 1 showing a linker solution added to a fourth interaction cell;

FIG. 8 is a graphical illustration of the embodiment of FIG. 1 showing a wash solution added to a first interaction cell;

FIG. 9 is a graphical illustration of the embodiment of FIG. 1 showing a ligand solution added to a first interaction cell;

FIG. 10 is a graphical illustration of the embodiment of FIG. 1 showing a buffer solution added to a first interaction cell;

FIG. 11 is a graphical illustration of the embodiment of FIG. 1 showing a sample solution added to a first interaction cell;

FIG. 12 is a graphical illustration of the embodiment of FIG. 1 showing a sample solution added to a second interaction cell;

FIG. 13 is a graphical illustration of the embodiment of FIG. 1 showing a sample solution added to a third interaction cell;

FIG. 14 is a graphical illustration of the embodiment of FIG. 1 showing a sample solution added to a fourth interaction cell;

FIG. 15 is a graphical illustration of an apparatus for performing microfluidic analysis in accordance with another embodiment of the invention;

FIG. 16 is a graphical illustration of the embodiment of FIG. 15 showing a solution added to a first interaction cell;

FIG. 17 is a graphical illustration of the embodiment of FIG. 15 showing a solution added to a second interaction cell;

FIG. 18 is a graphical illustration of the embodiment of FIG. 15 showing a solution added to a third interaction cell;

FIG. 19 is a graphical illustration of the embodiment of FIG. 15 showing a solution added to a fourth interaction cell;

FIG. 20 is a schematic flow chart illustrating a fluidics system for use in accordance with a method for identifying an analyte in a plurality of sample fluids in accordance with a further embodiment of the invention;

FIG. 21 is a drawing showing an embodiment of a component of the microfluidics platform which is an exemplary cantilever chip;

FIG. 22 is a drawing showing an engineering plan showing exemplary dimensions for the cantilever chip of FIG. 21;

FIG. 23 is a graph showing data obtained from tracings of cantilever movement and apparent reference plane movement data shown in nanometers (m, on the ordinate) as a function of time (in seconds on the abscissa), in response to thermal effects;

FIG. 24 is a graph showing data obtained from tracings of cantilever movement and data showing apparent reference plane movement as a function of time, in response to viscosity effects associated with different flow rates during sample addition;

FIG. 25 is a graph showing data obtained from tracings of cantilever movement and reference plane data as a function of time during formation of a self-assembled monolayer (SAM) on a gold surface of a cantilever;

FIG. 26 is a drawing of perspective views of components of an exemplary microfluidics housing or cartridge which includes a heating element. FIG. 26A is a drawing of the cartridge lid. FIG. 26B is a drawing of the window. FIG. 26C is a drawing of the cantilever chip of FIGS. 21 and 22. FIG. 26D is a drawing of the cartridge base with a pocket for the chip;

FIG. 27 is a drawing of a bottom view of the window of FIG. 26B;

FIG. 28 is a drawing of the window exploded, showing perspective views of the layers comprising the window of FIGS. 26B and 27. FIG. 28A is a drawing of a two-panel support layer. FIG. 28B is a drawing of the via layer. FIG. 28C is a drawing of the channel layer. FIG. 28D is a drawing of the transparent window substrate layer;

FIG. 29 is a drawing of a top view of the lid of the cartridge shown in FIG. 26A;

FIG. 30 is a drawing of a breakout view of the cartridge; and

FIG. 31 is a drawing of a cross-section view of the cartridge.

### DETAILED DESCRIPTION OF SPECIFIC EMBODIMENTS

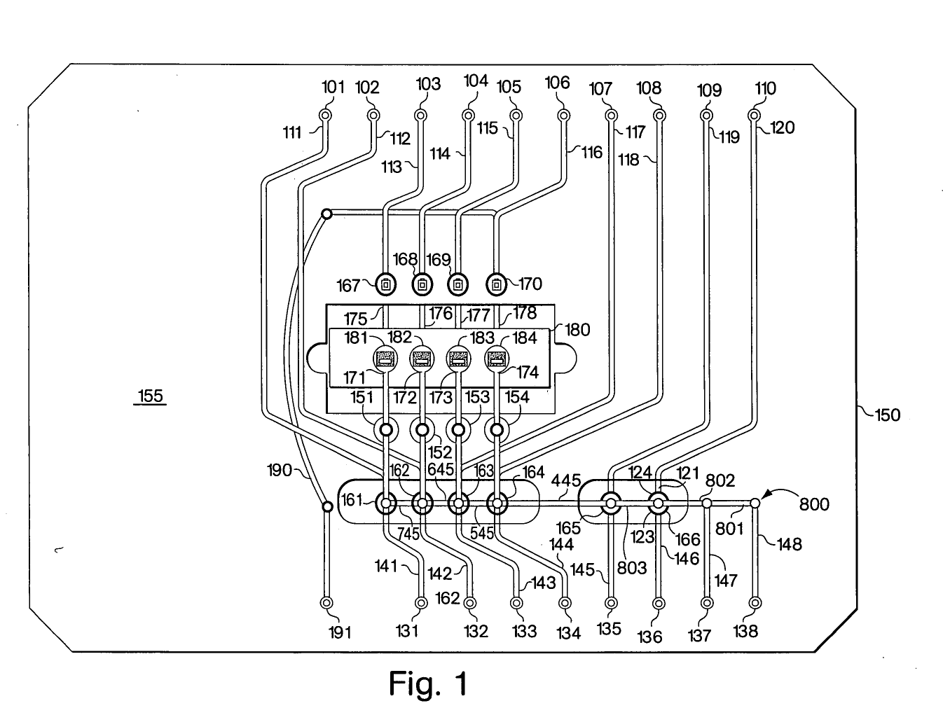

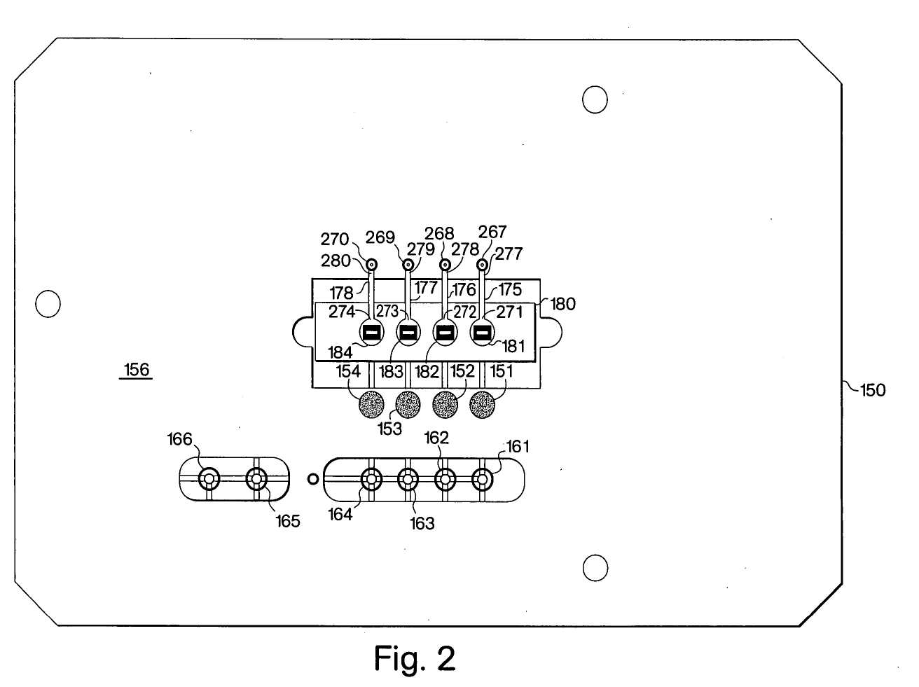

FIG. 1 is a graphical illustration showing a bottom view of an apparatus for performing microfluidic analysis in accordance with an embodiment of the invention. The apparatus includes three-dimensional housing 150 having a plurality of fluid lines 141-148. The fluid lines 141-148 are disposed within the housing in at least two layers such that some fluid lines are closer to a top face of the housing 155 and others are closer to a bottom face of the housing 156, shown in FIG. 2. Each of the fluid lines has an inlet 131-138 for receiving a fluid from a fluid pump or other fluid delivery apparatus. Such a fluid pump may be external to the housing 150 or it may be part of the housing so as to create a completely self-contained unit. The housing 150 also includes a plurality of control lines 111-120 in communication with valves 161-170. Valves 161-166 are in communication with the fluid lines 141-148. Each of the control lines 111-120 receives a control fluid, such as a gas or other fluid, from an inlet 101-110. The fluid lines 141-148, control lines 111-120 and fluid paths (discussed below) may be about 0.5 mm in diameter. For example, the diameter of the lines and paths may range from about 0.05 mm to about 0.6 mm. In accordance with further embodiments of the invention, the diameter of the lines and paths may be about 0.05 mm to about 0.2 mm; from about 0.1 mm to about 0.3 mm; and from about 0.2 mm to about 0.6 mm. Control fluid and other fluids may be provided to the apparatus through the use of a robotic device, or may be provided manually.

A plurality of valves 161-170 control the flow of fluid into and out of a microcantilever platform 180. In this embodiment, the valves 161-166 are two-way valves that communicate with the fluid lines 141-148. The valves 161-166 all lead to a common line or manifold 800 comprising fluid paths 801-803 and 445, 545, 645, and 745, and each valve has an input and an output. For example, valve 166 has an input 121 for receiving control fluid from control line 120 and an output 124 that permits fluid to flow both from fluid line 146 and fluid path 802. In other words, valve 166 controls the output of fluid line 146 as well as the output of fluid path 802, which runs under fluid line 146. As shown in FIG. 2, valves 167-170 are also two-way valves and each of these has a valve inlet 267-270 and a valve outlet 277-280. In order for fluid to flow though the housing, at least one of valves 167-170 must be open.

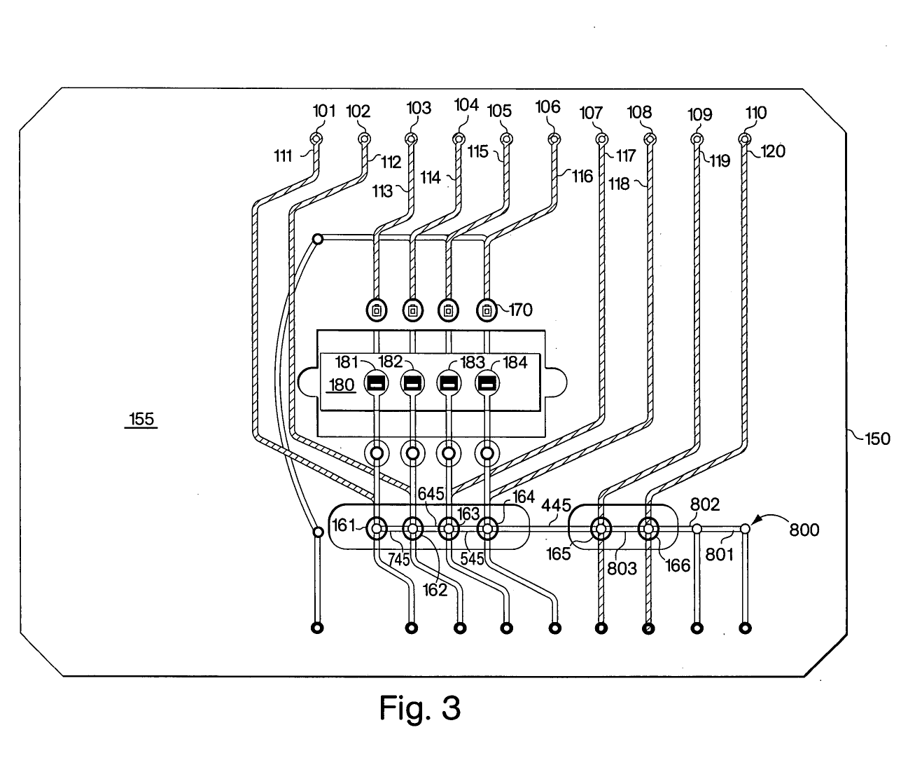

The valves 161-170 may be pneumatic valves that are activated by the control fluid. In the embodiment of FIGS. 1-14, the control fluid, when pressurized, serves to close the valves 161-170. In FIG. 1 the control fluid has not been pressurized, thus the valves are all open, whereas in FIG. 3, the control fluid is pressurized and the valves 161-170 are closed. When the control fluid is a high density gas, such as air, the response time of the valves quickens. The number valves in the apparatus may be less than, more than or equal to the number of fluid lines. Similarly, the number of valves may be less than, equal to or more than the number of control lines.

The microcantilever platform 180 is disposed in the housing 150 and includes a plurality of interaction cells 181-184. Each of the interaction cells 181-184 has an inlet 171-174 for receiving one or more preparation fluids and a sample fluid and an outlet, 271-274 as shown in FIG. 2, for releasing fluid from the cell through output lines 175-178. The interaction cells may be about 4 mm in diameter. For example, the diameter of the interaction cells may range from about 0.5 mm to about 6 mm. In accordance with further embodiments of the invention, the diameter of the interaction cells may range from about 0.5 mm to about 2.5 mm; from about 1 mm to about 3 mm; from about 2 mm to about 5 mm; or from about 3 mm to about 6 mm.

In accordance with an embodiment of the invention, the microcantilever platform 180 is a micro-mechanical system wherein each of the interaction cells includes at least one microcantilever configured to deflect in response to interactions with a chemical component of the sample fluid. Alternatively, each of the interaction cells 181-184 may include a plurality of microcantilevers provided in a planar array of fingers.

As used herein, the term “microcantilever” or “cantilever” is a structural term that refers to a flexible beam that may be bar-shaped, V-shaped, or have other shapes, depending on its application. One end of the microcantilever may be fixed on a supporting base with another end standing freely. Microcantilevers are usually of microscopic dimensions, for example, they can be about 50 μm to about 750 μm in length. In accordance with an embodiment of the invention, the microcantilevers are 200 μm to 700 μm in length, 250 μm to 600 μm in length, or 300 μm to 500 μm in length. Cantilevers can further be at least 750 μm in length (750 microns) or 0.75 mm (millimeters) to 1.0 mm, or 0.75 mm to 1.25 mm, or 1.0 mm to 1.5 mm in length. Further, the width can be, for example, about 50 μm to about 300 μm. Each microcantilever may be from about 0.5 μm to about 4.0 μm thick. Silicon and silicon nitride are the most common molecules used to fabricate microcantilevers. However, other molecules may be used for making cantilevers, including piezoelectric molecules, plastic molecules and various metals.

In accordance with embodiments of the invention, the microcantilevers can be manufactured from ceramics, silicon, silicon nitride, other silicon compounds, metal compounds, gallium arsenide, germanium, germanium dioxide, zinc oxide, diamond, quartz, palladium, tantalum pentoxide, and plastic polymers. Plastics can include: polystyrene, polyimide, epoxy, polynorbornene, polycyclobutene, polymethyl methacrylate, polycarbonate, polyvinylidene fluoride, polytetrafluoroethylene, polyphenylene ether, polyethylene terephthalate, polyethylene naphthalate, polypyrrole, and polythiophene. Microcantilevers that are custom fabricated may be obtained from, for example, Diffraction Ltd., Waitsfield, V T. Further, U.S. Pat. No. 6,096,559 issued Aug. 1, 2000, and U.S. Pat. No. 6,050,722 issued Apr. 18, 2000, describe fabrication of a microcantilever, including use of material such as ceramics, plastic polymers, quartz, silicon nitride, silicon, silicon oxide, aluminum oxide, tantalum pentoxide, germanium, germanium dioxide, gallium arsenide, zinc oxide, and silicon compounds.

Microcantilevers that can be employed in accordance with the invention may have a compound immobilized on the surface of a free end to detect and screen receptor/ligand interactions, antibody/antigen interactions and nucleic acid interactions as is disclosed in U.S. Pat. No. 5,992,226, issued on Nov. 30, 1999. Microcantilevers can be used to detect enzyme activities directed against a substrate located on a surface of the microcantilever. Deflection may be measured using either optical (see U.S. patent application Ser. No. 10/70,434 filed Nov. 10, 2003, published Aug. 26, 2004 as 2004/0165244A1, the entire contents of which are expressly incorporated herein by reference) or piezoelectric methods. Further, the microcantilevers of the embodiments of the invention can measure concentrations using electrical methods to detect phase difference signals that can be matched with natural resonant frequencies as shown in U.S. Pat. No. 6,041,642, issued Mar. 28, 2000. Determining a concentration of a target species using a change in resonant properties of a microcantilever on which a known molecule is disposed, for example, a biomolecule selected from DNA, RNA, and protein, is described in U.S. Pat. No. 5,763,768.

In accordance with embodiments of this invention, a method and apparatus for detecting and measuring physical and chemical parameters in a sample media may use micromechanical potentiometric sensors as disclosed in U.S. Pat. No. 6,016,686, issued Jan. 25, 2000. Chemical detection of a chemical analyte is described in U.S. Pat. No. 5,923,421, issued Jul. 13, 1999. Further, magnetic and electrical monitoring of radioimmune assays, using for example, antibodies specific for target species which cause microcantilever deflection (e.g., magnetic beads binding the target to the microcantilever, as described in U.S. Pat. No. 5,807,758, issued Sep. 15, 1998) is consistent with embodiments of the invention.

The term “first surface” as used herein refers to that geometric surface of a microcantilever designed to receive and bind to a ligand and further to an analyte. One or more coatings can be deposited upon this first surface. The term “second surface” refers to the area of the opposite side of the microcantilever that is designed not to receive the ligand or bind to the analyte.

As the second surface is generally not coated, it is generally comprised of the material from which the microcantilever or microcantilever array is fabricated, prior to any coating procedure applied to the first surface. Alternatively, it may be coated with a material different from the first surface's coating.

Coating of micromechanical sensors with various interactive molecules is described in U.S. Pat. No. 6,118,124, issued Sep. 12, 2000. A coating material is deposited on a microcantilever by depositing a metal which may be selected from at least one of the group consisting of aluminum, copper, gold, chromium, titanium, silver, and mercury. Further, a plurality of metals may be deposited on a microcantilever by depositing, for example, a first layer of chromium and a second layer of gold, or a first layer of titanium and a second layer of gold. Coatings may be amalgams or alloys comprising a plurality of metals.

In accordance with embodiments of the invention, a first surface of a microcantilever can be fabricated to have an intermediate layer, for example, sandwiched between the first surface comprising for example, gold, and the second surface, comprising for example silicon nitride. The intermediate layer may be an alloy comprising a plurality of metals. For example, the intermediate layer may be an amalgam comprising mercury with at least one of chromium, silver, and titanium.

A microcantilever may deflect or bend from a first position to at least a second position due to differential stress on a first surface of the microcantilever in comparison to a second surface. That is, a microcantilever may deflect in response to the change in surface stress or surface tension resulting from exposure of the microcantilever to a component of a particular environment. A microcantilever may also deflect in response to a change in the environment. A change in the environment may occur as the result of adding a sample having or lacking an analyte, having a higher or lower analyte concentration, adding or omitting a specific co-factor of an analyte, having a higher or lower concentration of the co-factor, having or lacking a specific inhibitor of an analyte, or having a higher or lower concentration of an inhibitor. Further, a sample may be diluted or concentrated and a solution may experience a change in temperature, pH, conductivity or viscosity prior to, during or after exposure to a microcantilever.

When one end of a microcantilever is fixed to a supporting base as described above, deflection is measured by measuring a distance the distal end of the microcantilever (i.e., the end distal to the end fixed to the supporting base) has moved. The distal end may move from a first position to a second position. In the first position, the biomaterial on the first surface of the microcantilever has not yet bound to or reacted with the analyte. In the second position, the biomaterial on the microcantilever has bound to or has reacted with the analyte in the environment.

A “deflection characteristic”, as used herein, is a pattern of deflection of a microcantilever that is reproducible in extent of distance traveled, for example as measured in nm (nanometers) and/or frequency per unit time, for example oscillations per unit time. The deflection characteristic can distinguish specific conditions of ligand and analyte, and further reaction conditions such as temperature, concentration, ionic strength, presence of cation or other co-factors, preservatives, and other conditions well known to one of the chemical arts. The deflection under these conditions thereby can become a signature for the specific reaction. A deflection characteristic is calculated from a measurement of movement of the microcantilever upon addition of a sample, or measurement of movement as a function of concentration of an analyte, a ligand, an inhibitor, or a co-factor. A deflection characteristic may also be calculated as a function of pH, or of temperature, and the like.

Each of the interaction cells 181-184 may receive a different sample fluid as will be discussed in more detail below. A microprocessor can be included in an apparatus or a method, such that an integrated circuit containing the arithmetic, logic, and control circuitry required to interpret and execute instructions from a computer program may be employed to control a mechanism for fluid distribution; such as activation of the valves. Further, microprocessor components of the measuring devices may reside in an apparatus for detection of microcantilever deflection.

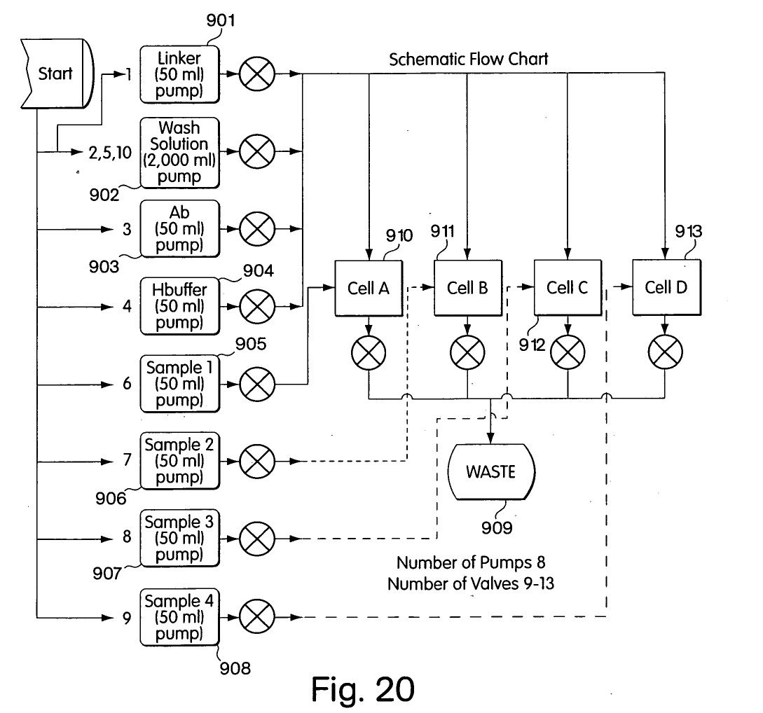

The apparatus may also include a plurality of expansion chambers 151-154 for eliminating gas from fluids entering the interaction chamber 181-184, and a waste line 190 with a waste outlet 191 for releasing waste from the interaction cells 181-184 into a waste receptacle (item 909 in FIG. 20). Further, each interaction cell 181-184 may be in fluid communication with its own waste receptacle or with a reservoir for collecting the contents of the interaction cell in order to perform further analysis on what is contained in the reservoir. Further analysis may include gel electrophoresis, and the gel electrophoresis may be multi-dimensional. Additionally, at least one of the dimensions may be polyacrylamide gel electrophoresis in the presence of a denaturing detergent.

Further analysis may also include mass spectroscopy.

The apparatus of FIGS. 1 and 2 may be a card or cartridge consisting of about 17 layers of one or more plastic polymers. Such cards and cartridges may be custom manufactured, for example, by Micronics, Inc. of Redmond, Wash. These cards or cartridges may be mounted in a manifold that receives fluid pump lines or fluid from other fluid delivery devices. Similarly, the pumps may be part of the card as mentioned above. The apparatus may also be mounted on a temperature-controlled platform. The apparatus may be used to identify a particular molecule in one or more sample fluids, as is shown in FIGS. 3-14.

In an alternative embodiment, the apparatus has an assembly top and bottom which, when combined, form a set of chambers or interaction cells, and fluid lines, in a housing. As in embodiments above, the apparatus may be mounted on a temperature-controlled platform, an analyzed by means of an opto-mechanical assembly, as described in U.S. patent application Ser. No. 10/705,434 filed Nov. 10, 2003 and incorporated by reference herein in its entirety. A novel feature of the present invention provided herein is a cartridge having a top and bottom which not only form the chambers and fluid lines, but include a heating element such that fluid inlet lines pass through the device and acquire the temperature of the cartridge.

In FIG. 3, a control fluid, in this case a gas such as air, is pressurized in the control lines 111-120 through the inlets 101-110 to close the valves 161-170. When all the valves have been closed, fluid cannot flow into or out of the interaction cells. Thus, all of the valves are initially closed (by pressurizing a control fluid in the control lines) and then opened (by de-pressurizing the control fluid) to allow liquid to flow into appropriate interaction cells. This is done so that preparation fluids, such as linker, buffer, ligand solutions, and sample solutions containing an analyte may be input to the interaction cells 181-184 in a discriminatory manner. For example, a buffer solution may be input to all of the cells or to a subset of the cells, for example, to three of the cells, two of the cells or only to one of the cells. Similarly, a different sample solution may be input to each of the cells, or to a subset of the cells.

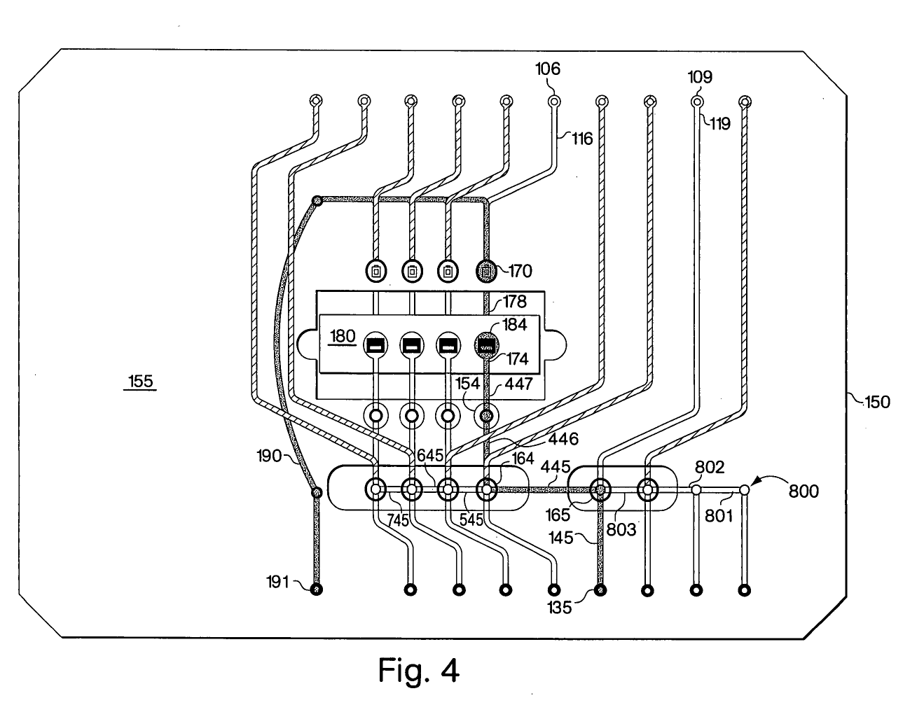

FIG. 4 shows a linker solution being added to a first interaction cell. The term “linker solution” may include the following compounds: dithiobis(succinimidyl-undecanoate) (DSU), which can be purchased from Pierce Endogen, Inc. (Rockford, Ill.); long chain succinimido-6[3-(2-pyridyldithio)-propionamido]hexanoate (LCSPDP), which contains pyridyldithio and NHS ester reactive groups that react with sulfhydryl and amino groups and can be purchased from Pierce; succinimidyl-6[3-(2-pyridyldithio)-propionamido]hexanoate (SPDP), which contains pyridyldithio and NHS ester reactive groups that react with sulfhydryl and amino groups and can be purchased from Pierce; and m-maleimidobenzoyl-N-hydroxysuccinimide ester (MBS), which contains NHS ester and maleimide reactive groups that react with amino and sulfhydryl groups, and can be purchased from Pierce.

To add linker to the first interaction cell, inlets 109 and 106 do not receive the control gas, thus no control gas is input to control lines 119 and 116, and valves 165 and 170 are opened. The linker solution flows from a fluid pump or other fluid delivery device to inlet 135 into fluid line 145. Since valve 165 is open, the fluid may then flow through fluid path 445 into fluid path 446, and into expansion chamber 154. Gas may optionally be eliminated from the linker solution in the expansion chamber 154, and the linker solution flows through fluid path 447 into interaction cell 184 via inlet 174. Any outflow of fluid from the interaction cell 184 will flow into output line 178, and because valve 170 is open, the outflow will be stored in a waste receptacle (or in a reservoir for collection) via fluid waste line 190 and waste outlet 191.

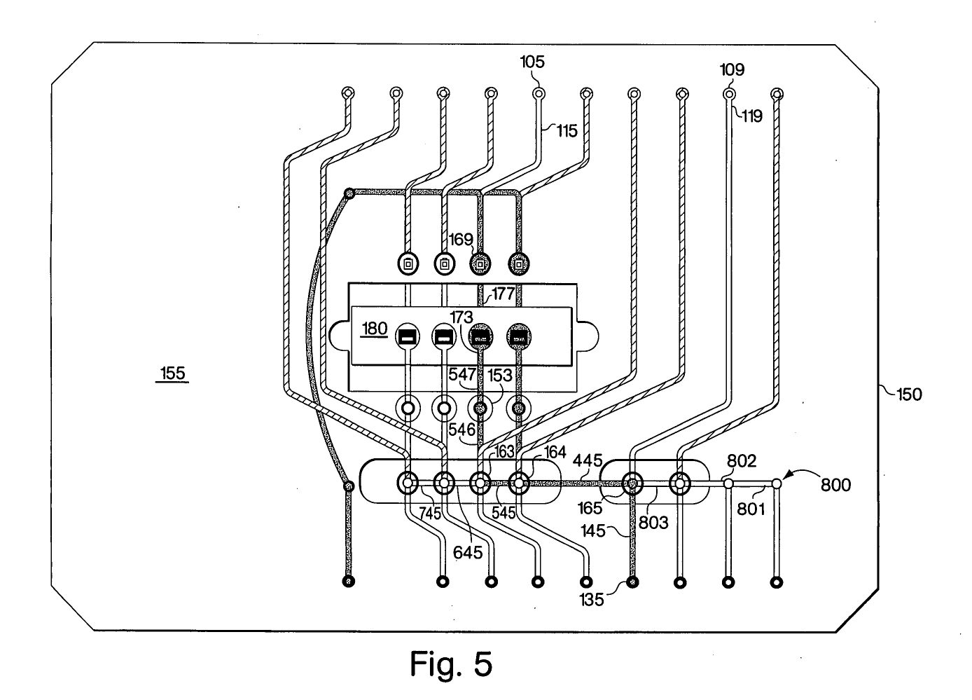

FIG. 5 illustrates how the linker solution may be added to the second interaction cell, while keeping all other interaction cells isolated, by pressurizing the control fluid in all control lines except control lines 115 and 119, thus opening valves 165 and 169. As above, the linker solution flows from a fluid pump to inlet 135 into fluid line 145 and then through fluid paths 445 and 545. Note that the control lines 111, 112, 117, and 118 intersect fluid lines 141, 142, 143 and 144 respectively at a point above valves 161-164.

Consequently, fluid may flow from fluid path 445 to 545 in a relatively unrestricted manner. At this point the fluid will flow into fluid path 546, and then into expansion chamber 153. Gas is eliminated from the linker solution in the expansion chamber 153, and the linker solution flows through fluid path 547 into interaction cell 183 via inlet 173. Any outflow of fluid from the interaction cell 183 will flow into output line 177, and because valve 169 is open, the outflow will be stored in a waste receptacle via fluid waste line 190 and waste outlet 191.

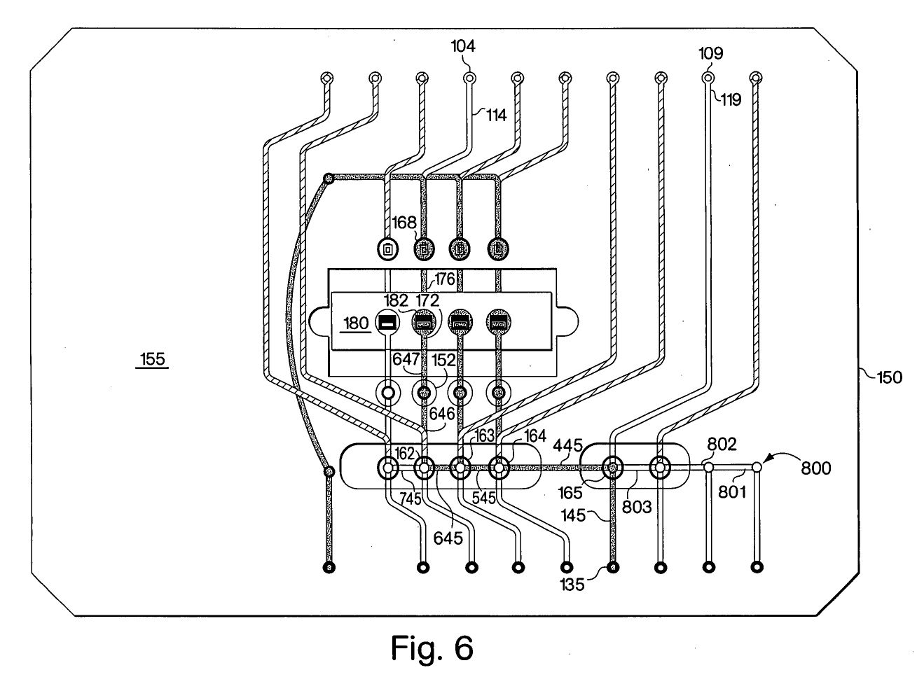

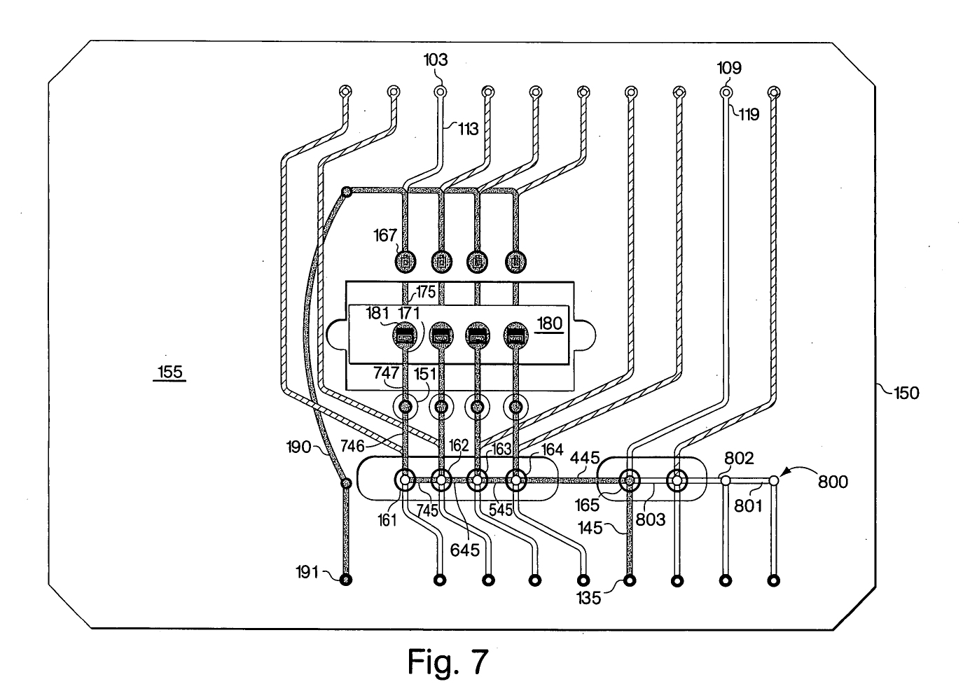

FIGS. 6 and 7 show the linker solution being added to interaction cells 182 and 181 respectively. In accordance with this embodiment, each interaction cell 182 and 181 will receive the linker solution while all other cells are isolated in the manner described above with respect to FIGS. 4 and 5. To add the linker solution to interaction cell 182, control fluid will not be pressurized in control lines 114 and 119, causing valves 165 and 168 to open. Linker solution will flow from a fluid pump to inlet 135 into fluid line 145 and then through fluid paths 445, 545, and 645. The fluid will then flow into fluid path 646, and into expansion chamber 152. Gas will be eliminated from the linker solution in the expansion chamber 152, and the linker solution will flow through fluid path 647 into interaction cell 182 via inlet 172. Outflow of fluid from the interaction cell 182 will flow into output line 176, and because valve 168 is open, the outflow will be stored in a waste receptacle via fluid waste line 190 and waste outlet 191.

To add the linker solution to interaction cell 181, control fluid is not pressurized in control lines 113 and 119, causing valves 165 and 167 to open. Linker solution will flow from a fluid pump to inlet 135 into fluid line 145 and then through fluid paths 445, 545, 645, and 745. The fluid will then flow into fluid path 746, and into expansion chamber 151. Gas will be eliminated from the linker solution in the expansion chamber 151, and the linker solution will flow through fluid path 747 into interaction cell 181 via inlet 171. Outflow of fluid from the interaction cell 181 will flow into output line 175, and because valve 167 is open, the outflow will be stored in a waste receptacle via fluid waste line 190 and waste outlet 191.

The linker solution may be added to a subset of the plurality of interaction cells, or to all of the interaction cells, illustrated here for exemplary purposes only as four of the cells 181, 182, 183 and 184, by opening valve 165 with valves 167, 168, 169 and 170 simultaneously. Similarly, any subset of interaction cells may receive linker solution simultaneously by opening valve 165 and the valves that correspond to the interaction cells to be filled. Further, waste line 190 may lead to a plurality of reservoirs, and the outflow from the interaction cells may be stored in respective reservoirs for further analysis.

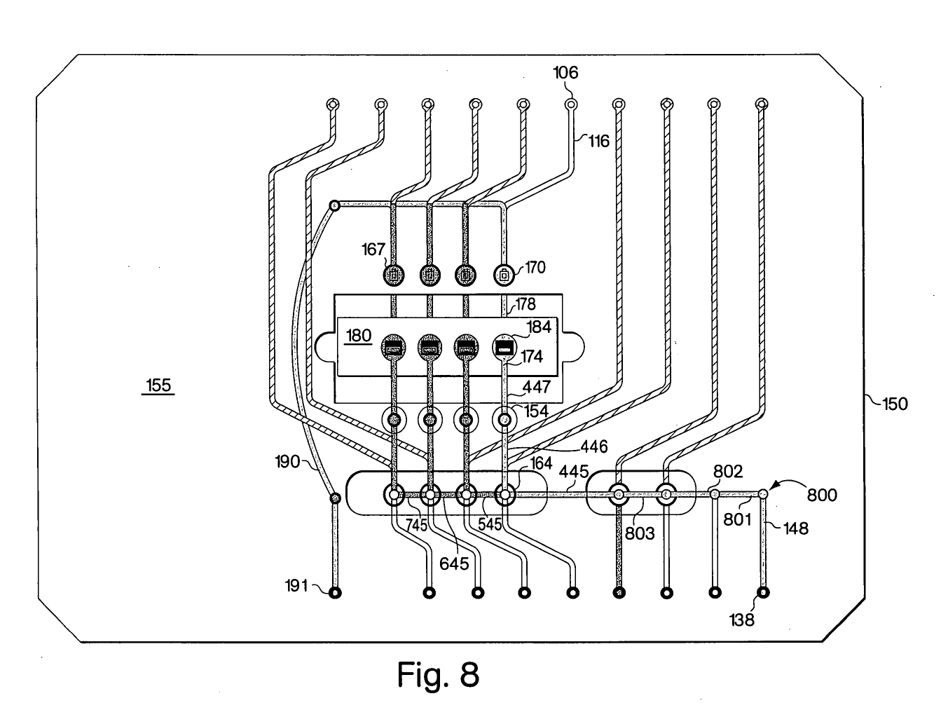

Valves may be provided to insure that outflow from each interaction cell is stored in its corresponding reservoir. Alternatively, reservoir lines and outlets may be provided for each interaction cell, rather than one line and outlet (such as waste line 190 and outlet 191). FIG. 8 is a graphical illustration of the embodiment of FIG. 1 showing a wash solution added to a first interaction cell. The wash solution will flow from a fluid pump or other fluid delivery device to fluid line 148 via inlet 138. In accordance with this embodiment, no control line is in direct communication with fluid line 148 (though such a control line could be provided) thus, only control line 116 is de-pressurized. Valve 170 is opened, allowing the wash solution to flow into fluid path 445 via fluid paths 801-803. From this point, the wash process continues as described above with respect to the linker solution and FIG. 4-7, to provide each interaction cell with the wash solution.

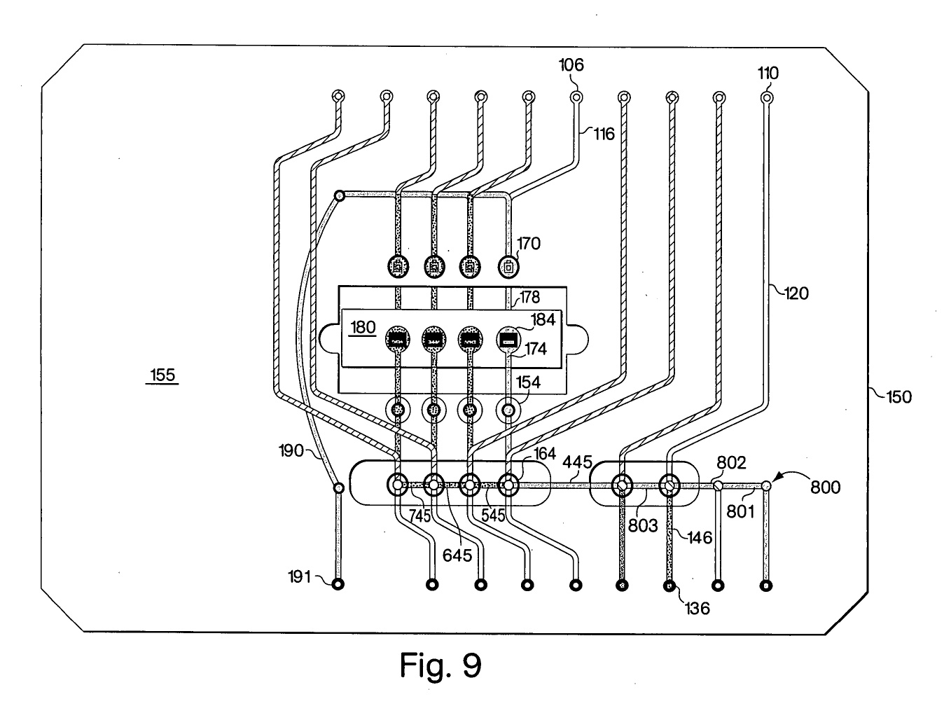

FIG. 9 is a graphical illustration of the embodiment of FIG. 1 showing a ligand solution, for example, an antibody solution, added to a first interaction cell. The ligand may react chemically with previously applied linker molecules. The ligand solution flows from a fluid pump or other fluid delivery device to fluid line 146 via inlet 136. Control lines 116 and 110 are de-pressurized and valves 170 and 166 are opened, allowing the ligand solution to flow into fluid path 445 via fluid path 803. From this point, the ligand solution proceeds through the apparatus as described above with respect to the linker and wash solutions to provide each interaction cell with the ligand solution.

In an alternative embodiment, a cantilever chip is manufactured, and then emplaced in the bottom portion of a cartridge that forms the “floor” or bottom of the plurality of interaction cells, such that subsequent emplacement of the top of the cartridge on the bottom, as described herein, forms the microfluidics device. In this embodiment the cartridge is reusable, the plurality of cantilevers in each of the plurality of interaction cells are made more uniform, and many issues of manufacture are resolved. Cantilevers may be modified by use of cross-linkers as above, to attach a ligand for an analyte, prior to placement in the interaction cell and assembly by addition of the cartridge top. Alternatively, cantilevers are modified by use of cross-linkers following emplacement of the cantilever chip within the cartridge by any of the exemplary cross-linking reagents described herein. Increased specificity of modification is achieved by methods described in U.S. patent application Ser. No. 10/847,755 filed May 18, 2005, published Nov. 17, 2005, and which is incorporated herein by reference in its entirety.

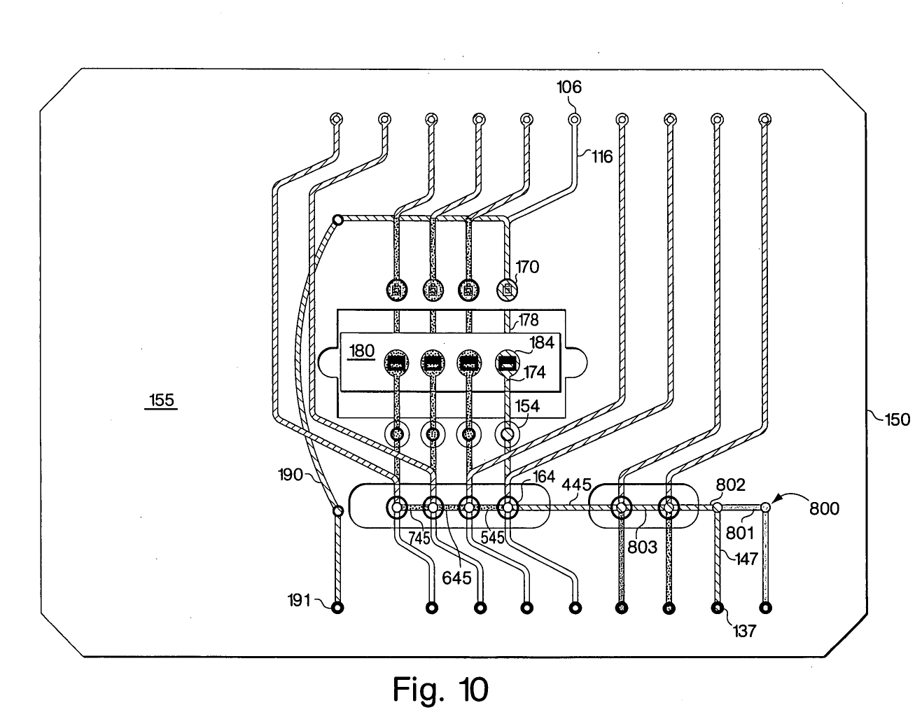

FIG. 10 is a graphical illustration of the embodiment of FIG. 1 showing a buffer solution added to a first interaction cell. The buffer solution flows from a fluid pump or other fluid delivery device to fluid line 147 via inlet 137. As was the case with the wash solution, no control line is in direct communication with fluid line 147 (again, such a control line could be provided), thus only control line 116 is de-pressurized. Valve 170 is opened, allowing the buffer solution to flow into fluid path 445 via fluid paths 802-803. From this point, the buffer solution proceeds to each interaction cell in accordance with the embodiments of FIGS. 4-7. A wash process may follow the addition of the buffer solution to each cell and will proceed as described above with respect to FIG. 8.

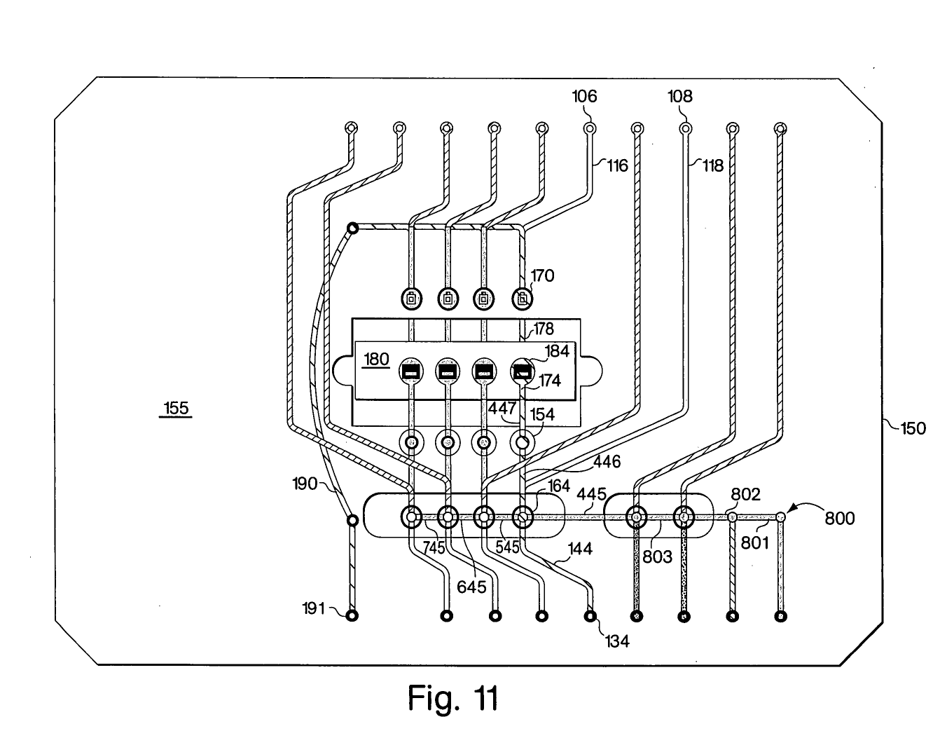

In FIG. 11, a first sample solution having an analyte, or a control solution, is added to the first interaction cell. The first sample solution flows from a fluid pump or other fluid delivery device to fluid line 144 via inlet 134. Control lines 116 and 118 are de-pressurized and valves 170 and 164 are opened, allowing the first sample solution to flow into fluid path 446 and into expansion chamber 154. From this point, the first sample solution proceeds to the interaction cell 184 via fluid path 447 and inlet 174. Outflow of the first sample solution from the interaction cell 184 will flow into output line 178, and the outflow will be stored in a waste receptacle (or reservoir for collection) via waste line 190 (or reservoir line) and waste outlet (or reservoir outlet) 191.

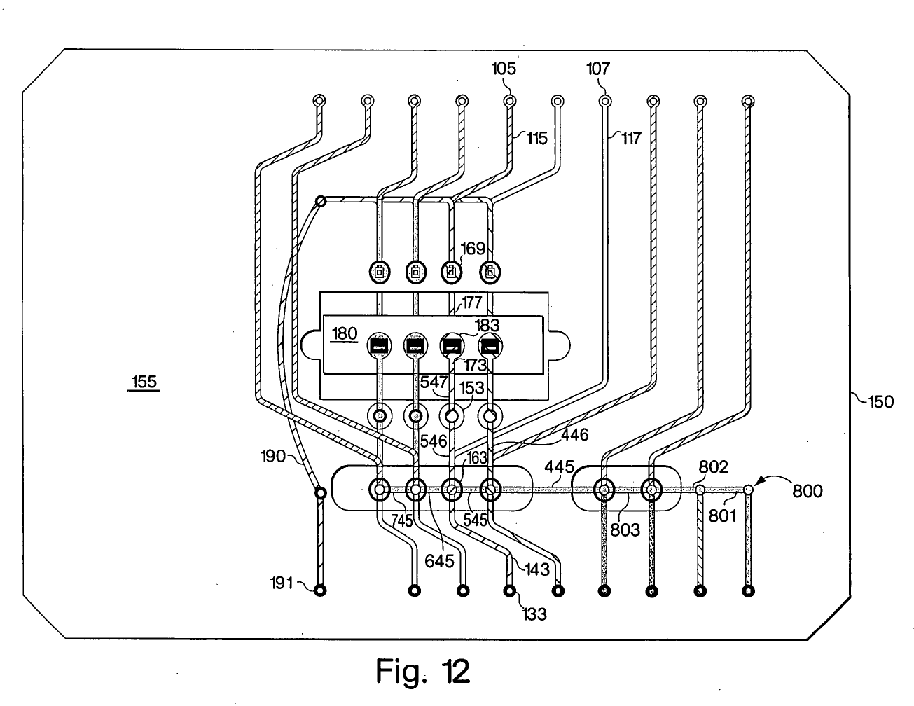

FIG. 12 is a graphical illustration showing that the second interaction cell may be provided with a second sample solution. To provide interaction cell 183 with the second sample solution, control lines 115 and 117 are de-pressurized, valves 169 and 163 are opened and the second sample solution flows from a fluid pump to fluid line 143 via inlet 133. The second sample solution will flow into fluid path 546 and into expansion chamber 153. The second sample solution proceeds to the interaction cell 183 via fluid path 547 and inlet 173. As above, outflow of the second sample solution from the interaction cell 183 will flow into output line 177, and the outflow will be stored in a waste receptacle (or reservoir for collection) via waste line 190 and waste outlet 191.

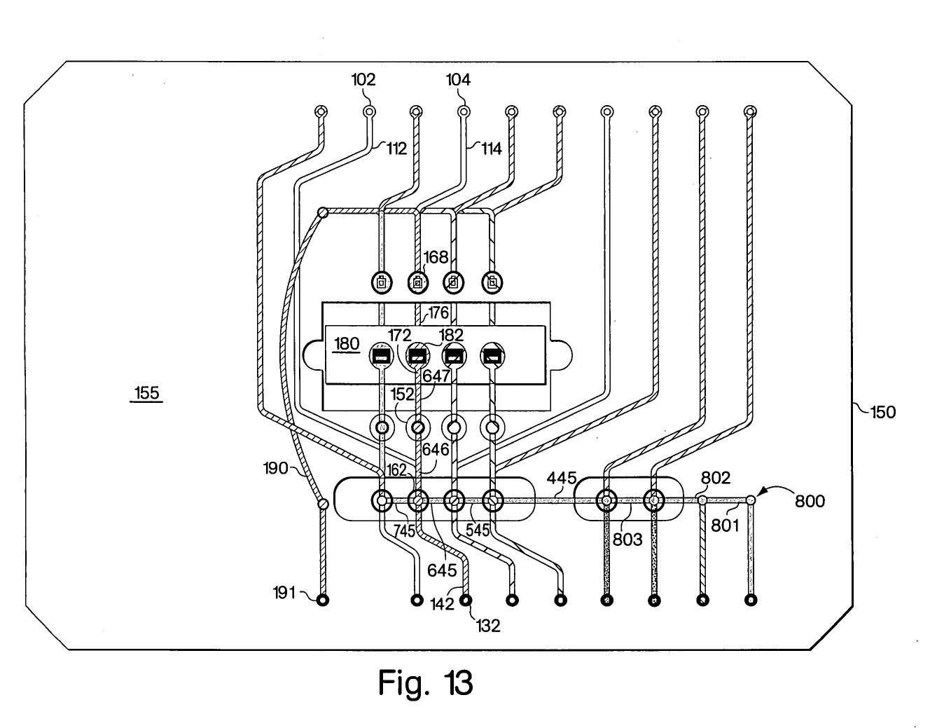

FIG. 13 illustrates a way to provide the third interaction cell 182 with a third sample solution, control lines 112 and 114 are de-pressurized, valves 162 and 166 are opened and the third sample solution flows from a fluid pump to fluid line 142 via inlet 132. The third sample solution will flow into fluid path 646 and into expansion chamber 152 and gas will be removed from the solution. The third sample solution proceeds to the interaction cell 182 via fluid path 647 and inlet 172. Outflow of the third sample solution from the interaction cell 182 will flow into output line 176, and the outflow will be stored in a waste receptacle (or reservoir for collection) via waste line 190 and waste outlet 191.

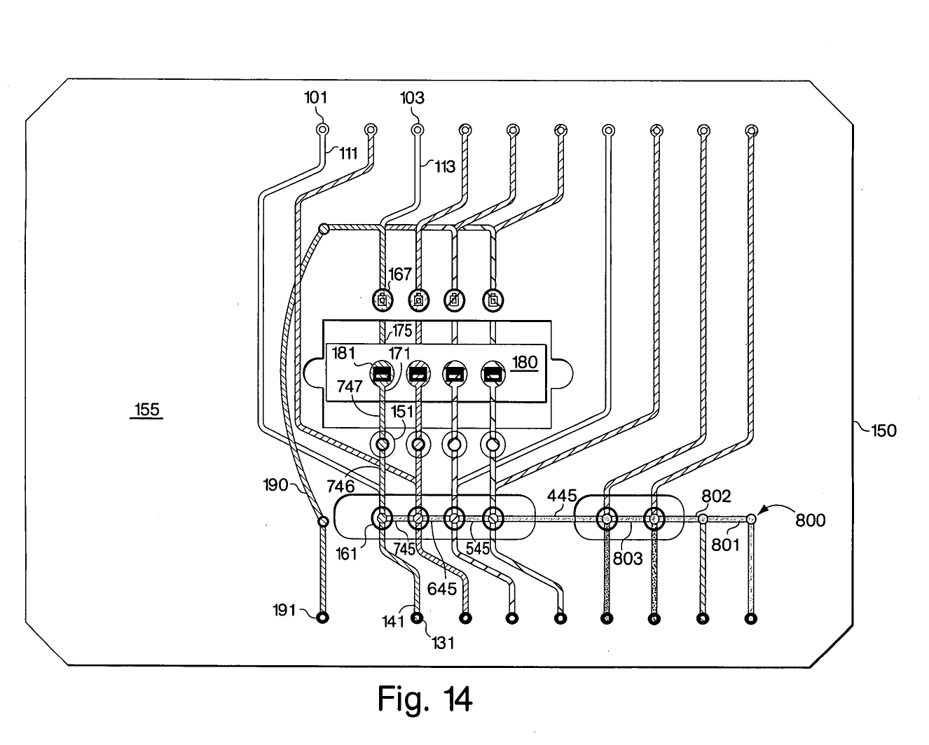

FIG. 14 illustrates a way to provide the fourth interaction cell 181 with a fourth sample solution. Here, control lines 111 and 113 are de-pressurized, valves 161 and 167 are opened and the fourth sample solution flows from a fluid pump to fluid line 141 via inlet 131. The fourth sample solution will flow into fluid path 746 and into expansion chamber 151, and gas will be removed from the solution. The third sample solution proceeds to the interaction cell 181 via fluid path 747 and inlet 171. Outflow of the third sample solution from the interaction cell 181 will flow into output line 175, and the outflow will be stored in a waste receptacle (or reservoir for collection) via waste line 190 and waste outlet 191.

Each of the interaction cells includes at least one microcantilever, or an array of microcantilevers, configured to deflect in response to chemical interactions with a component of the sample fluid. In a particular embodiment of the invention, a planar array of microcantilever fingers is disposed in each interaction cell such that one or more microcantilever finger deflects with respect to the plane of the array in response to a reaction with a molecular component of the sample solution.

In an alternative embodiment, an aliquot of each of a plurality of sample solutions is added to each of a corresponding plurality of interaction cells by a pump mechanism using the lines formed in the cartridge having a top and bottom, each of the top and bottom contributing a component of the fluid lines, after emplacement of the cantilever chip into the cartridge bottom and enclosure by emplacement of the top.

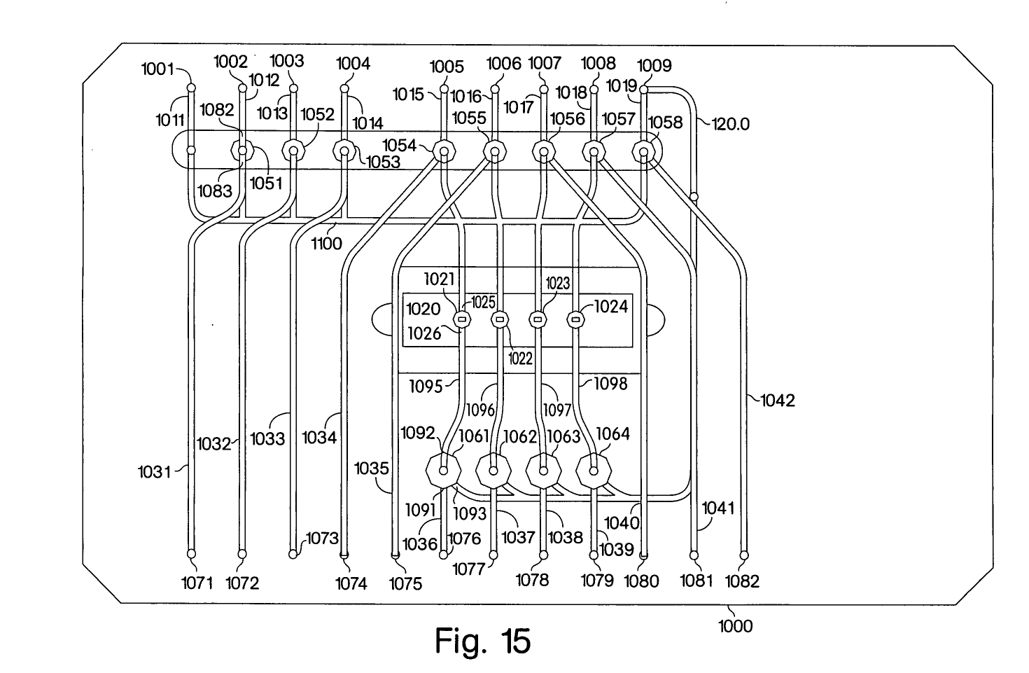

FIG. 15 is a graphical illustration showing an apparatus for performing microfluidic analysis in which the valves of the apparatus are normally closed in accordance with another embodiment of the invention. Unlike the embodiment of FIG. 1, in FIG. 15, all of the valves 1051-1058 and 1061-1064 are closed under normal atmospheric pressure (when only air is in the lines). This configuration reduces the duty cycle of the electrical components of the system and minimizes the amount of current needed to drive the system. However, whether the valves are open or closed under normal atmospheric conditions is purely arbitrary and each of the embodiments of FIG. 1 and FIG. 15 may operate either way with respect to the configuration of lines and valves. Additionally, in accordance with the embodiment of FIG. 15, the fluid lines 1011-1019 and fluid inlets 1001-1008 are at the top of the figure, rather than at the bottom as in FIG. 1.

The apparatus includes a three-dimensional housing 1000 having a plurality of fluid lines 1011-1019. Each of the fluid lines 1011-1018 has an inlet 1001-1008 for receiving a fluid from a fluid pump or other fluid delivery apparatus. The housing 1000 also includes a plurality of control lines 1031-1042 in communication with the fluid lines 1011-1019. Each of the control lines 1031-1042 receives a control fluid from an inlet 1071-1082. The fluid lines 1011-1019, control lines 1031-1042 and fluid paths of this embodiment may be dimensioned in a manner similar to the fluid lines, control lines, and fluid paths described with respect to FIG. 1. Here again, control fluid and other fluids may be provided to the apparatus through the use of a robotic device, or may be provided manually.

The plurality of valves 1051-1058 and 1061-1064 control the flow of fluid into and out of a microcantilever platform 1020. The valves may be two-way valves that function as three-way valves as described above with respect to the embodiment of FIG. 1. Thus, each valve has an inlet and an outlet. For example, valve 1051 has a valve inlet 1082 for receiving the control fluid from control line 1031, a valve outlet 1083 for transmitting fluid from fluid line 1012 to the manifold 1100. The valves 1051-1058 and 1061-1064 are activated (in this case opened) by the control fluid. As above, when the control fluid is a high density gas the response time of the valves quickens. The valves may be activated or deactivated under control of a computer program resident on a microprocessor. Further, the number valves in the apparatus may be less than, more than or equal to the number of fluid lines. Similarly, the number of valves may be less than, equal to or more than the number of control lines.

The microcantilever platform 1020 is disposed in the housing 1000 and includes a plurality of interaction cells 1021-1024. Each of the interaction cells 1021-1024 has an inlet, such as 1025, for receiving one or more preparation fluids and a sample fluid and an outlet, such as 1026, for releasing fluid from the cell through output lines 1095-1098.

The apparatus of FIG. 15 may further include a waste line 1200 with a waste outlet 1009 for releasing waste from the interaction cells 1021-1024 into a waste receptacle. As was the case with the embodiment of FIG. 1, each interaction cell 1021-1024 may be in fluid communication with its own waste receptacle or with a reservoir for collecting the contents of the interaction cell in order to perform further analysis on what is contained in the reservoir.

As was the case with the apparatus of FIG. 1, the embodiment of FIG. 15 may be in the form of a card or cartridge comprising one more plastic polymers. Preparation fluids, such as linker, buffer, ligand solutions, and sample solutions may be input to the interaction cells 1021-1024 in a discriminatory manner. A buffer solution may be input to all of the cells or to a subset of the cells, for example, to three of the cells, two of the cells or only to one of the cells. Similarly, a different sample solution may be input to each of the cells, or to a subset of the cells.

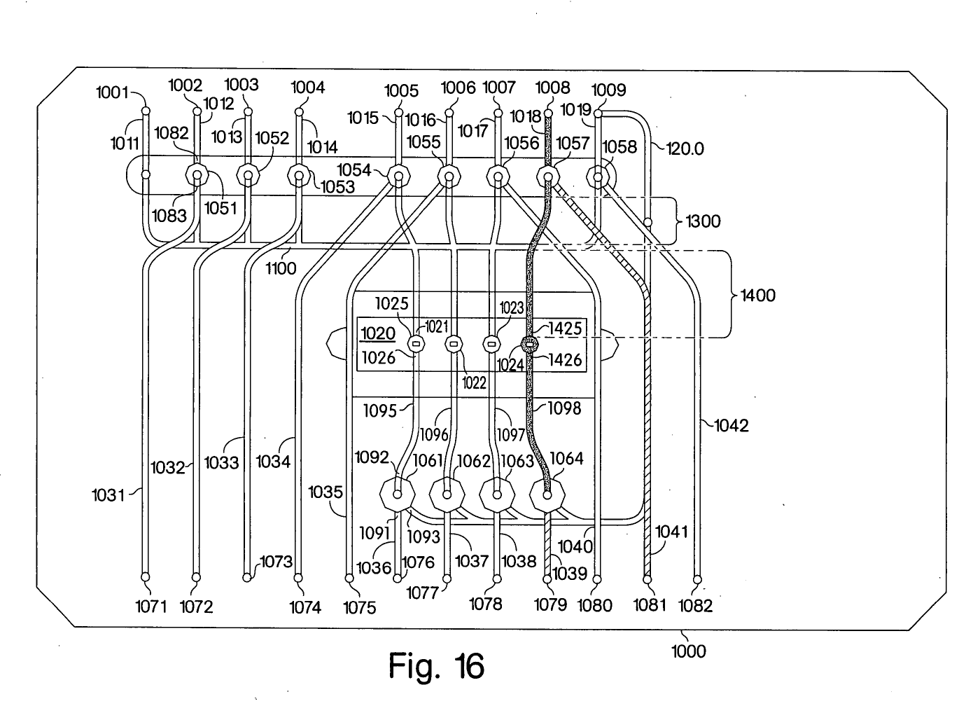

FIG. 16 shows a solution being added to a first interaction cell. To accomplish this, inlets 1081 and 1079 receive the control gas, thus control gas is input to control lines 1041 and 1039 respectively, and valves 1057 and 1064 are opened. The solution flows from a fluid pump or other fluid delivery device to inlet 1008 into fluid line 1018. Since valve 1057 is open, the fluid may then flow through fluid path 1300 into fluid path 1400, and into interaction cell 1024 via inlet 1425. Any outflow of fluid from the interaction cell 1024 will flow into output line 1098, and because valve 1064 is open, the outflow will be stored in a waste receptacle (or in a reservoir for collection) via fluid waste line 1200 and waste outlet 1009.

The solution may be added to a subset of the plurality of interaction cells, or to all of the interaction cells, illustrated here for exemplary purposes only as four cells. Similarly, any subset of interaction cells may receive a solution simultaneously by opening the valves that correspond to the appropriate interaction cells to be filled (as will be evident from the descriptions FIGS. 16-20). Further, the waste line 1200 may lead to a plurality of reservoirs, and the outflow from the interaction cells may be stored in respective reservoirs for further analysis. Valves may be provided to insure that outflow from each interaction cell is stored in its corresponding reservoir. Alternatively, reservoir lines and outlets may be provided for each interaction cell, rather than one line and outlet.

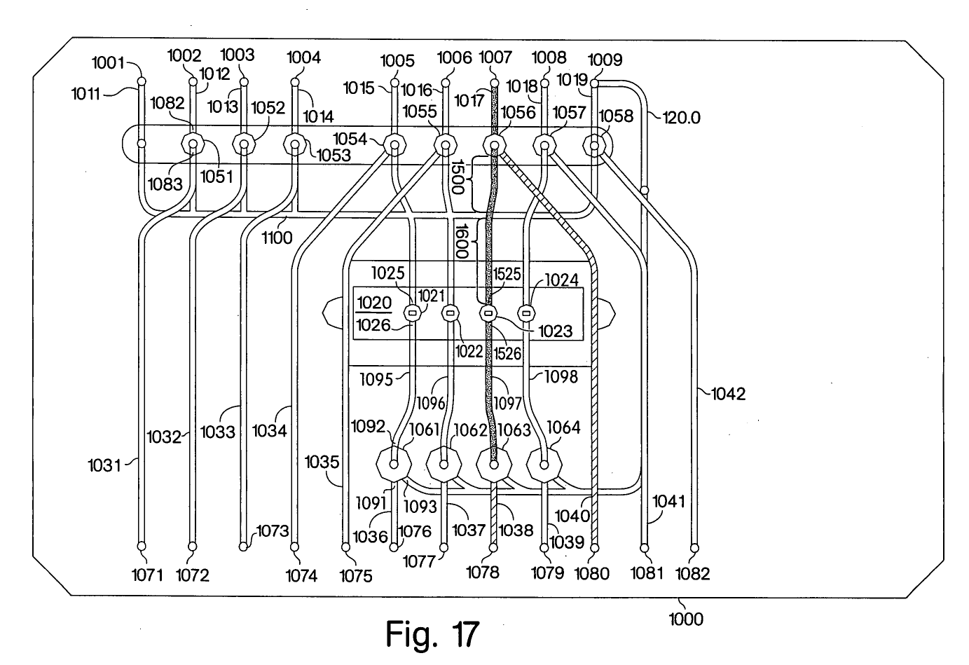

FIG. 17 is a graphical illustration of the embodiment of FIG. 15 showing a solution added to a second interaction cell. Here, inlets 1080 and 1078 receive the control gas, thus control gas is input to control lines 1040 and 1038 respectively, and valves 1056 and 1063 are opened. The solution flows to inlet 1007 into fluid line 1017. Since valve 1056 is open, the fluid may then flow through fluid path 1500 into fluid path 1600, and into interaction cell 1023 via inlet 1525. Any outflow of fluid from the interaction cell 1023 will flow into output line 1097, and because valve 1063 is open, the outflow will be stored in a waste receptacle or reservoir via fluid waste line 1200 and waste outlet 1009.

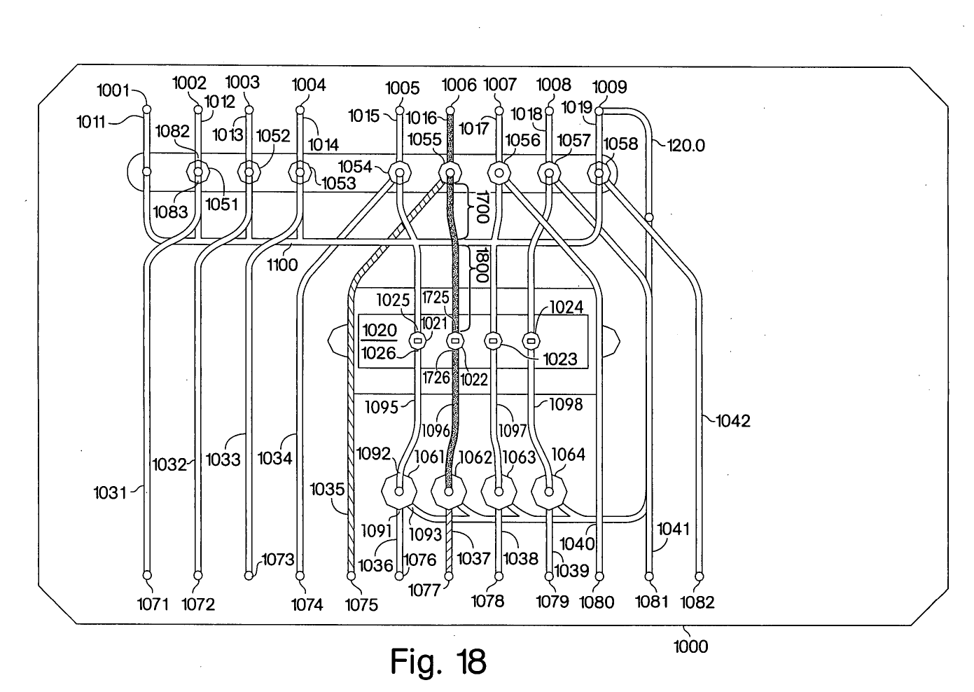

FIG. 18 is a graphical illustration of the embodiment of FIG. 15 showing a solution added to a third interaction cell. Inlets 1075 and 1077 receive the control gas, and control gas is input to control lines 1035 and 1037 respectively. Valves 1055 and 1062 are opened. The solution flows from a fluid pump or other fluid delivery device to inlet 1006 into fluid line 1016. Since valve 1055 is open, the fluid may then flow through fluid path 1700 into fluid path 1800, and into interaction cell 1022 via inlet 1725. Again, outflow of fluid from the interaction cell 1022 will flow into output line 1096, and because valve 1062 is open, the outflow will be stored via fluid waste line 1200 and waste outlet 1009.

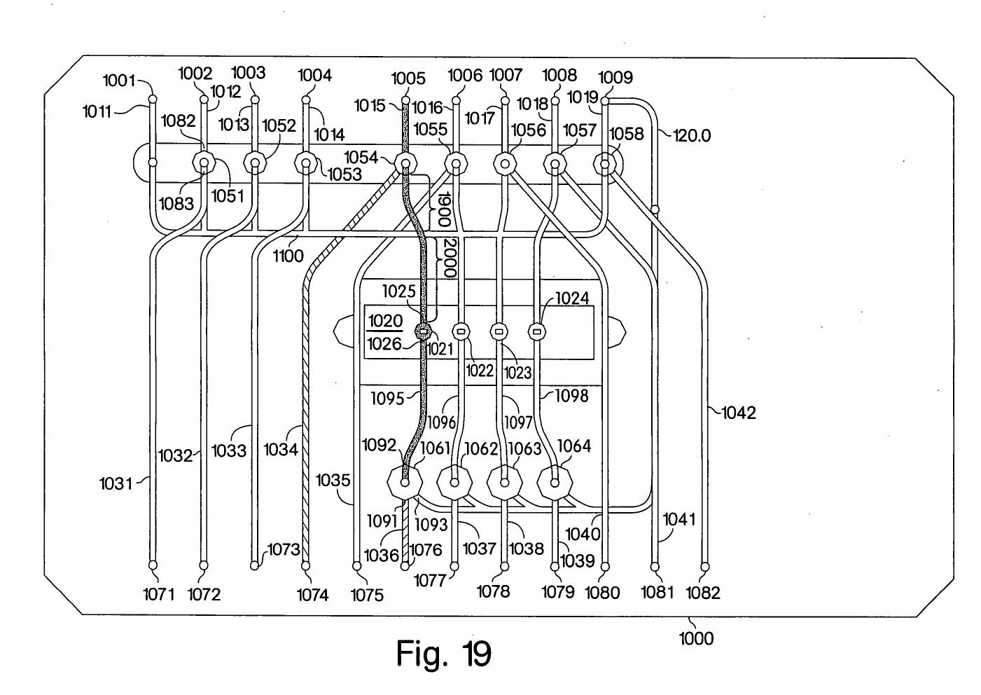

FIG. 19 is a graphical illustration of the embodiment of FIG. 15 showing a solution added to a fourth interaction cell. Inlets 1074 and 1076 receive the control gas, which is input to control lines 1034 and 1036 respectively. Valves 1054 and 1061 are opened, and the solution flows from to inlet 1005 into fluid line 1015. Since valve 1054 is open, the fluid may then flow through fluid path 1900 into fluid path 2000, and into interaction cell 1021 via inlet 1225. Any outflow of fluid from the interaction cell 1021 will flow into output line 1095, and because valve 1061 is open, the outflow will be stored in a waste receptacle or reservoir via fluid waste line 1200 and waste outlet 1009.

FIG. 20 is a schematic flow chart illustrating fluidics system for use in accordance with a method for identifying an analyte in a plurality of sample fluids in accordance with a further embodiment of the invention. In accordance with this embodiment, one or more preparation solutions 901-904 are input into one or more of a plurality of interaction cells. At least one of the preparation fluids includes a ligand that has affinity for the analyte. Each interaction cell includes at least one microcantilever such that the ligand binds to the microcantilever. At least one sample solution 905-908 is input into one or more of the interaction cells, and a deflection of the microcantilever is detected in each sample solution containing the analyte. In one embodiment, the device may be mounted in a manifold and/or on a temperature-controlled platform. Outflow from the interaction cells 910-913 may be stored in a waste receptacle 909 or in a reservoir for further analysis as described above.

One of the preparation fluids may be a solution of a linker 901 capable of covalently linking the ligand, here defined as the material affixed to a surface of the microcantilever, to the microcantilever. Another preparation fluid may be a wash solution 902, and the wash solution may be input to one or a plurality of the interaction cells one or more times. Yet another preparation fluid may be a ligand or a “receptor” solution 903, i.e., a biological macromolecule known to have affinity for a specific binding portion, or a ligand for a class of analytes. The receptor can also be a ligand for an analyte, the presence and/or amount of which is to be detected in one or in a series of sample. Another preparation fluid may be a buffer solution 904. The number of sample solutions may equal the number of interaction cells or the number of sample solutions may be less than the number of interaction cells.

The ligand may be a biomaterial, for example, a protein such as an enzyme or a synthetic polypeptide, or it can be a nucleic acid such as RNA or DNA. A biomaterial that is a macromolecule may comprise all or a portion of a nucleic acid or a protein. The protein or polypeptide may comprise an epitope, an antibody, an antibody fragment, an enzyme, or any other embodiment of a molecule containing peptide bonds. The analyte to be detected or quantified in a sample may be a biomaterial such as a macromolecule, or an organic or inorganic small molecule. Similarly, the analyte may be hormone, for example, the hormone may be a steroid for example, a sex steroid or a glucocorticoid, or a polypeptide hormone such as a cytokine. Either of the ligand or the analyte may comprise all or a portion of an antibody or an antigenic material, or all or a portion of an enzyme.

The ligand may be a substrate for an embodiment of the analyte which is an enzyme. In an alternative embodiment, the ligand may be an enzyme, and the analyte may be a substrate capable of interacting with the enzyme bound to a surface of a cantilever. See U.S. patent application Ser. No. 09/951,131 filed Sep. 12, 2001, published Apr. 10, 2003, 2003/0068655, and U.S. patent application Ser. No. 10/346,443 published Feb. 12, 2004, 2004/0029108, the entire contents of each of which are expressly incorporated herein by reference.

Examples and methods for the use of the apparatus of the invention are shown in Table 1. In Example 1, the apparatuses of FIGS. 3-19 is used to demonstrate a movement or deflection of a plurality of microcantilevers in a microcantilever array when a sample solution contains an analyte, such as a particular chemical or biological component, capable of binding to or interacting with a ligand affixed to a surface of the microcantilever. Cell A can be a reaction cell that provides a positive control; deflection of microcantilevers is caused by interaction on a surface of the cantilever of components of fluids sequentially provided to cell A. Cell B can be a reference cell; for example, a control buffer known to lack the analyte, is added to this cell instead of a sample. This control can determine the extent of microcantilever deflection that occurs as a result of interactions between preparation liquids such as a linker solution and an antibody solution, or other environmental forces. Cell C can be a negative control cell, for example, which has not been exposed to linker solution. Microcantilever deflection in this cell can determine the extent of ligand binding to a microcantilever surface directly, in the absence of a cross-lining agent. Cell D can be another control cell, containing for example, bovine serum albumin instead of the biomaterial of interest, so that microcantilever deflection is a measure of non-specific binding of the analyte.

In practice, measurement of quantities of an analyte in samples by cantilever deflection at quantitative levels is confounded by several sources of variability, including but not limited to: thermal variation within the opto-mechanical assembly; refractive index difference among sample fluids and between a sample fluid and a negative control; and local and temporal drift in electronics. While in theory these variables are minimized, it is desirable to have a method and apparatus to measure an environmental variation which can then be subtracted from a specific deflection or cantilever movement which is attributable solely to quantity of the analyte. Further, the means for this measure should be physically present in each interaction cell, so that local variation from cell to cell can be measured, and cantilever movement can accordingly be controlled.

Accordingly, an embodiment of the invention herein is a cantilever die or chip having a plurality of cantilever fingers and a reference plane. The cantilever fingers have a base and are all attached to the base, and have a free end that deflects in response to presence of an analyte in a sample in an environment, or in response to a change in configuration of a molecule present attached to the cantilever. In various embodiments of the present invention, the base comprises an area which is a reference plane and is contiguous to the area of the base of the cantilever fingers.

Deflection is actuated by the presence in the sample of an analyte, the cantilever fingers being configured to interact with the analyte by virtue of attachment of a ligand to one surface of the cantilevers. The ligand is also attached to the area of the reference plane. Detection of deflection is achieved by any of a variety of means, for example by detection of position of focused electromagnetic beams, by piezoelectric, or by piezomagnetic means. In each case the deflection or cantilever movement is converted to an electrical signal, separately generated for each cantilever, and transmitted to a computer.

For each cantilever chip or die contained in an interaction cell, environmental variables such as thermal variation within an opto-mechanical assembly, refractive index differences between samples or due to changes in flow rate, and drift in electronics, is measured by use of the reference plane portion of the die or chip. For example, when deflection is measured by angle of reflection of an electromagnetic beam focused on a cantilever, another electromagnetic beam is focused on the contiguous reference plane in the same interaction cell, so that in a sample having a high or a change in refractive index, a portion of a change in angle of reflection due to the refractive index is detected by the reference plane. This portion can be subtracted from the extent of apparent deflection measured for each contiguous cantilever.

The contents of all cited references are hereby incorporated by reference herein.

### EXAMPLE 1

In accordance with step 1 of Example 1 as illustrated in Table 1, the cross-linking agent DSU (dithiobis(succinimidylundecanoate)) in a volume of about 50 μl, is added to interaction cells A, B and D. DSU is a water soluble bifunctional cross-linking agent.

In step 2, as herein exemplified, all of the cells receive a wash solution in a volume of about 300 μl per cell. In step 3, all of the cells can receive about 50 μl of an antibody solution (such as an antibody specific for an oncogene protein such as Brc A or Wilm's Tumor, WT-1). A buffer having a low pH is provided to interaction cells, for example, to cells A, B and D, in a volume of about 50 μl per cell in step 4. This solution removes non-specifically bound material, i.e., those molecules of material which have not reacted with the cross-linking agent. Cells are washed with about 300 μl of the wash solution in step 5. In step 6, a volume of a sample solution containing, for example, an unknown quantity of a material that can interact with the antibody of step 3, for example, about 50 μl is provided to cells, for example, to cells A and C. A control material, e.g., bovine serum albumin is provided to cell D in a volume of about 50 μl in step 7. Cells are washed, for example, with about 300 μl of the wash solution in step 8.

Further in Example 1, it should be noted that the wash steps can be performed with the same solution, and that steps, for example, steps 6 and 7, can be preformed simultaneously. Further, any of the wash steps are optional in volume and timing; deflection of microcantilevers can be analyzed throughout, although measurement of deflection following steps 6 and 7 is most significant.

It is to be understood that a choice of a volume of fluid to use is merely suggested here and can be varied from the suggested amounts. Volumes for other use in the methods and apparatuses herein can be standardized within any given experiment according to a protocol to be devised by a user of ordinary skill in the art, and such alternative volumes are within the equivalents envisioned herein.

### EXAMPLE 2

Example 2 is an illustration of how the apparatus described herein may be used to identify a ligand in a plurality of sample solutions. Here cells A, B and C are reaction cells and cell D is used as a control cell. A volume, for example, of about 50 μl of DSU is provided to each cell in step 1. Next in step 2, a wash solution is provided to each cell in a volume of, for example, about 300 μl per cell. In steps 3 and 4, each cell is provided with about 50 μl of antibody solution and buffer solution respectively, and in step 5 the cells are subjected to another wash. A first sample solution, in a volume of about 50 μl, is then added to cell A in step 6. A second sample solution, also in volume of about 50 μl, is added to cell B in step 7, and a third sample solution of the same volume is added to cell C in step 8. It should be noted that in accordance with the apparatus described above, the first, second and third sample solutions may be provided to cells A, B and C, respectively, in one step. All of the cells are subjected to an optional wash process in step 9. Further, the solutions in one or more of the cells may be reused. That is, additional solutions may be added to one or more of the cells for further analysis.

### EXAMPLE 3

Example 3 illustrates how the apparatus described herein may be used to diagnose a patient simultaneously for one of a plurality of different viruses. Cells A, B and C are reaction cells and cell D is used as a control cell. A volume, for example, of about 50 μl of DSU is provided to each cell in step 1. In step 2, a wash solution is provided to each cell in a volume of, for example, about 300 μl per cell. In step 3 cell A is provided with about 50 μl of a first antibody solution. In step 4 cell B is provided with about 50 μl of a second antibody solution, and in step 5 cell C is provided with about 50 μl of a third antibody solution. Each antibody solution can have binding determinants directed against one of the viruses for the diagnosis. A volume of about 50 μl, of buffer solution is added to each of the cells step 6. All of the cells are then provided with about 300 μl of a wash solution in step 7, and in step 8 a volume of about 50 μl of a first, second, third sample solutions is provided to cells A, B, and C. The first, second and third antibody solutions may be provided to cells A, B and C, respectively, in one step. All of the cells can be subjected to an optional wash process in step 9.

### EXAMPLE 4

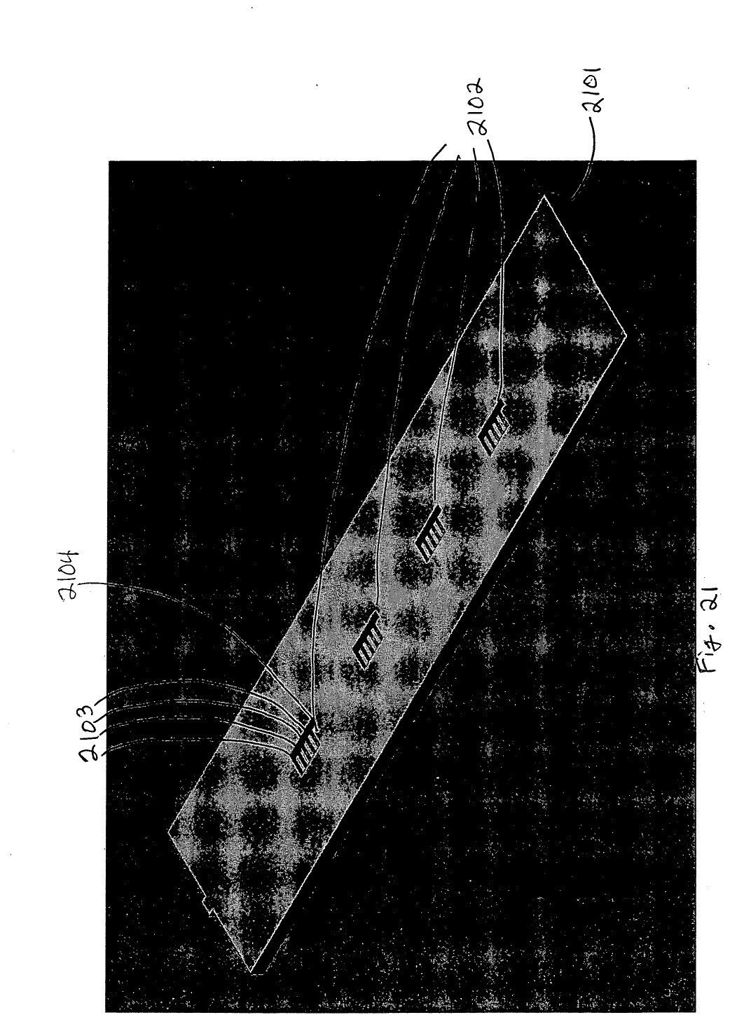

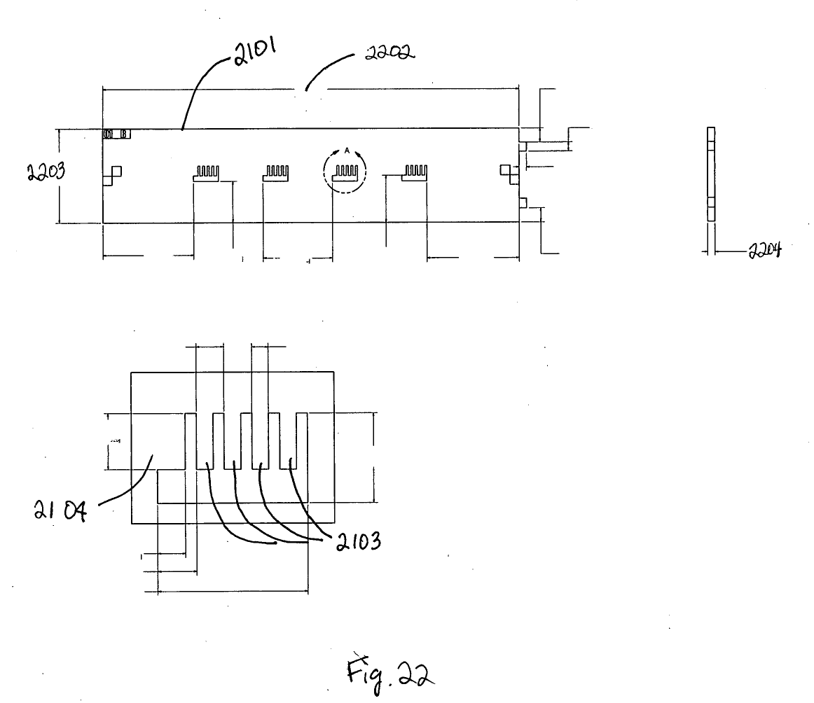

Reflection of a laser beam from a reference plane was used to control for thermal effects in the opto-mechanical assembly on which the microfluidics platform or chip is inserted, with the microfluidics platform or chip shown in FIGS. 21 and 22 in an embodiment of the invention.

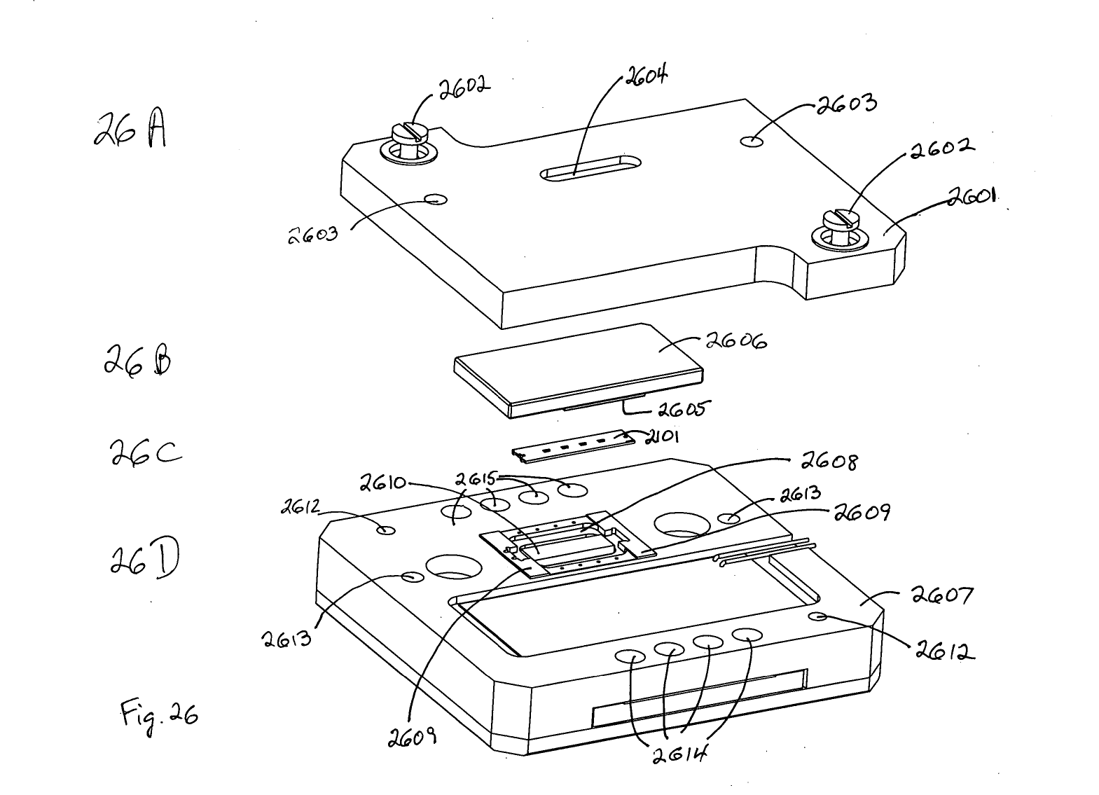

FIG. 21 is a drawing showing an embodiment of the microfluidics platform which is an exemplary microfluidics chip or die, 2101, having four sets, 2102 of four cantilevers, a single cantilever shown as 2103 for each set, the chip manufactured for emplacement into a microfluidics housing or cartridge. The chip or die sits on a surface, for example of Teflon@, and the cartridge shown in more detail in FIGS. 26-31, has a cover or lid with a gasket for retention of fluid volumes. Each set of cantilevers includes a reference plane, 2104. The portion of the chip in the vicinity of the cantilevers, 2102 in each set in combination with the surface and the lid form an interaction cell or well, for analysis of the presence of an analyte in the sample of fluid.

FIG. 22 is a drawing showing an engineering plan showing exemplary dimensions for the chip of FIG. 21, again showing the entire chip 2101, having four sets, 2102, of four cantilevers, each cantilever indicated as 2103. The relative and actual dimensions in the drawing are exemplary only and do not limit the scope of embodiments of the claims. Dimensions of the chip shown in this figure are 22.50 mm (millimeters) length, 2202 and 5.00 mm width 2303, and 0.38 height 2204 except where cut out for the cantilevers and reference plane, in the vicinity of which the height is 1 micron (micrometer or μm).

Dimensions of the embodiment of the cantilevers shown in this figure are 0.5 mm length (500 μm), 0.15 mm width (150 μm), and 0.001 height (1 μm). A gap of 0.10 mm (100 μm) corresponding to apparatus components for generating and focusing laser beams, is engineered as the distance between proximal edges of each cantilever and the next adjacent cantilever. The dimensions of the reference plane are: 0.50 mm (500 μm) length, corresponding to the lengths of the test cantilevers, and 0.25 mm (250 μm) width. Alternative embodiments tested with this design are cantilevers of 300 μm, 500 μm and 750 μm in length; and 0.5 μm and 1.0 μm in height. Dimensions are merely typical and are not limiting.

While variations in any of the dimensions of the chip from those shown in this example and in FIG. 22 are within the scope of the invention, the chip fits securely into a chip pocket of the cartridge illustrated in FIGS. 26-31, hence a chip having different dimensions, for use with a suitable cartridge with a chip pocket to accommodate those dimensions, are within the scope of the invention herein.

Detection of movement of the test cantilevers was achieved by converting position of a laser beam reflected from the cantilever surface into a current, using an array of one or more position sensitive detectors (PSD array). The detection system for generation and focusing of electromagnetic beams, in this case laser beams, and analysis of their reflection from the cantilevers relies on an opto-mechanical assembly, which is the entire ensemble of light source including its housing and mounting, the housing and mounting of the fluidics cartridge, beam splitters, cylindrical-lenses and the PSD array.

In general, the light source is a Vertical Cavity Surface Emitting Laser (VCSEL) chip, and as some of the components are closer to heat sources and sinks (hot spots and cooler spots, respectively), some of the cantilever components may move with respect to other components. Such movement is not ideal, and while it might be possible to eliminate movement due to thermal effects, such an instrument might also be more difficult to use. Alternatively the reference plane herein can provide information about apparent movement due to non-specific effects that might vary between each of the plurality of interaction cells in the chip, such as thermal effects.

Therefore the design of the cantilever chip herein enables a user an ability to monitor and measure such movement, and compensate by subtracting or normalizing actual known cantilever an apparent movement due to differences in thermal effects in the opto-mechanical assembly.

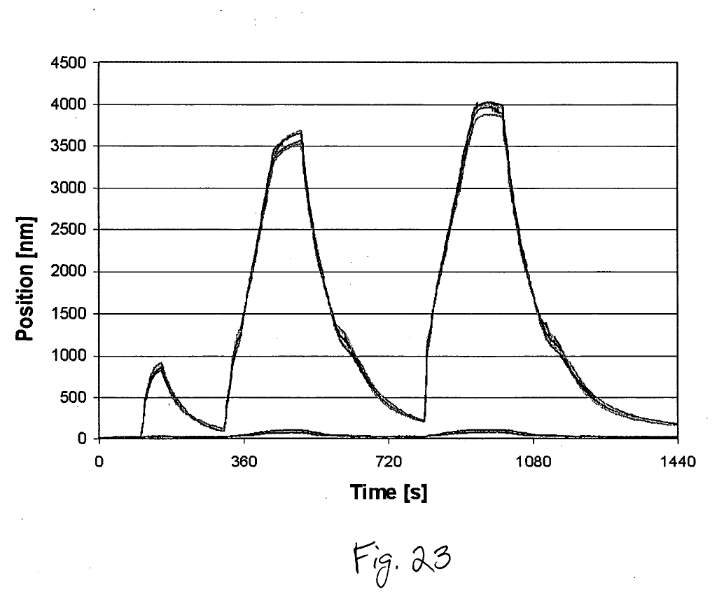

FIG. 23 is a graph showing data obtained from tracings of cantilever movement and apparent reference plane movement data shown in nanometers (m, on the ordinate) as a function of time (in seconds on the abscissa), in response to thermal effects. The tracings show cantilever positions for each of several cantilevers in at least one set, in comparison to data obtained from the reference plane. The heating unit of stage on which the chip was positioned was turned on and off each of three times, following which movement (cantilever bending) was detected by monitoring reflection of a laser beam focused on the cantilever or reference plane. The cantilever movement was detected at magnitudes of from about 900 nm to about 4000 nm. The apparent movement, seen as a change in reflection from the reference plane, was detected at positions of about 50 nm. The reference plane data is a control, and the magnitude is subtracted from the test or “actual” cantilever bending to obtain analyte specific data. The data seen in FIG. 23 shows that local effects of change in heat transferred from the heating element on apparent movement and from cantilever calorimetric effects can be determined and subtracted from data obtained due to analyte-ligand binding or due to conformational changes in the ligand attached to the cantilever, to obtain a true measure of extent of binding to the cantilever, or extent of conformational changes of a material bound to a cantilever.

FIG. 23 shows tracings from 4-6 test cantilevers due to movement of those cantilevers resulting from temperature changes. The cantilevers used are bimetallic, having a gold surface and a silicon layer. The switch for heating was turned on and off successively three times during the experiment, and large movements cause changes in positions of the test cantilevers of amplitude shown to be about 3500 nm. The test cantilevers display much greater movement than apparent movement of the reference plane, the tracings for which have an amplitude of less than about 100 nm.

### EXAMPLE 5

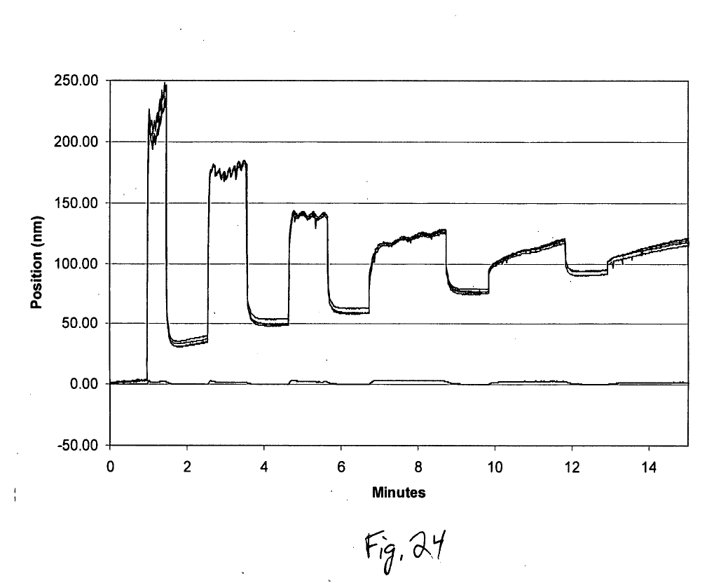

FIG. 24 is a graph showing data obtained from tracings of cantilever movement and data showing apparent reference plane movement as a function of time, in response to viscosity effects associated with different flow rates during sample addition. Sample was added to the interaction cells at each of the following flow rates, from the earliest time shown on the left of the abscissa to the latest time point on the right: 1280 microliters/min, 640 microliters/min, 320 microliters/min, 160 microliters/min, 80 microliters/min and 40 microliters/min. Extent of the changes in positions of the test cantilevers in response to the flow rate change, shown on the abscissa, is greater at higher flow rates (about 200-250 nm) than at lower flow rates (about 110-140 nm). Changes in apparent deflection of the reference plane are about the same (about 2-5 nm) under the various flow rates, i.e., are independent of the flow rate.

This example shows data obtained with use of cantilevers and a reference plane to control for changes in viscosity and flow rate. The switch for the pump controlling flow rate of buffer into the interaction cell was successively manipulated to provide changes to the flow rate into the interaction cells. The first peak at 1 min in FIG. 24 shows traces from movements of the test cantilever that responded to a flow rate of 1280 μl/min. Similarly, the smaller peak at about 2.5 min responded shows traces from movements of the test cantilever that responded to a flow rate of 640 μl/min. Further smaller and lower peaks at 3, 7 10 and 13 min were the observed responses to manipulating the flow rate to successive slower rates of 320, 160 80 and 40 μl/min. The data show also that the reference plane tracings respond to the changes in flow rate.

### EXAMPLE 6

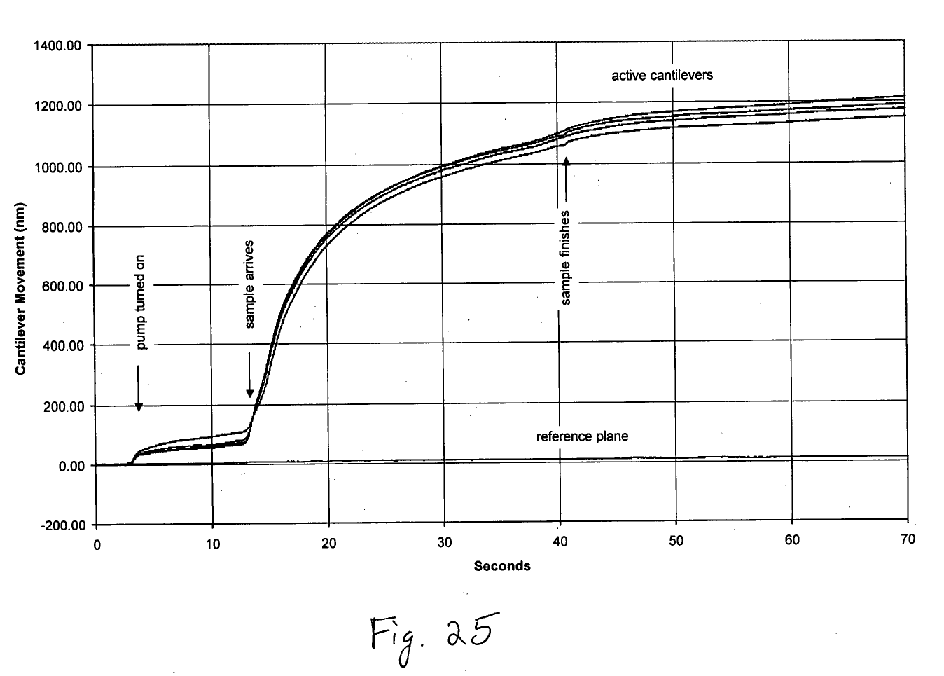

In this example movement of cantilevers was observed following addition of 4 mM 11-Mercapto-1-Undecanol in ethanol to interaction wells, causing formation of a self-assembled monolayer (SAM) and movement of cantilevers in response to changes in surface tension by formation of the SAM. The extent of cantilever movement shown in FIG. 25 at about 20 seconds following arrival of the sample in the interaction well at about 15 seconds was 800 nm, and more than 1000 nm when the sample finishes arriving. Further, the reference plane responds to formation of the SAM, however only to an extent of about 10-20 nm.

FIG. 25 is a graph showing data obtained from tracings of cantilever movement and reference plane data as a function of time during formation of a self-assembled monolayer (SAM) on a gold surface of a cantilever. At the time shown by arrow in the figure of about 13 seconds, labeled “sample arrives”, a sample of 4 mM solution of 11-Mercapto-1-Undecanol in ethanol was pumped into the interaction cell. Tracings show cantilever movement of the four test cantilevers, labeled “active”, of an amount of movement of magnitude greater than 1000 nm, approaching 1200 nm at the time point indicated by the arrow labeled, “sample finishes”. Also shown in FIG. 25 is apparent movement of the reference plane in response to sample addition, which is comparable to that seen in FIG. 24, i.e., very little deflection as measured by the tracing near 0.00. These data show that the cantilever and microfluidics system herein can detect changes in conformation of molecules and deposition onto the cantilever.

A set of cantilevers each having a gold surface, the cantilevers arranged to be contiguous to and co-planar with a reference plane as shown in FIG. 21, was exposed to a 4 mM solution of 11-Mercapto-1-Undecanol in ethanol. As a result of this exposure, a self assembled monolayer (SAM) was formed on the gold side of the cantilevers, as shown in FIG. 25. The formation of the SAM causes the cantilevers to bend away from the gold side (which is in the positive direction in the plot). The data include separate tracings from each of four cantilevers show that, in comparison to the four test cantilevers, the reference plane was essentially flat. These data indicate the significance of the test cantilever movements.

The reference plan functions also as a general diagnostic of overall apparatus function, i.e., an indicator that various aspects of the device are working together properly. The reference plane reads out a signal that is stable, robust and predictable in the absence of problems with the assay and the device.

### EXAMPLE 7

The above examples were performed and data obtained using another embodiment of the microfluidics platform herein, as shown in FIGS. 26-31, in which the housing of a re-useable microfluidics apparatus further includes a cartridge with a flow cell and a thermal control, i.e., an internal temperature sensor with electrical power source and heating means. The metal material from which the cartridge is any metal that is heat conducting and corrosion resistant, thus the cartridge is made from, for example, stainless steel, copper, and aluminum. A heating unit may be internal or external and in apposition to the cartridge, for maintaining the cartridge block at a uniform temperature, and for warming fluids as they pass through the channels and enter the wells to that temperature.

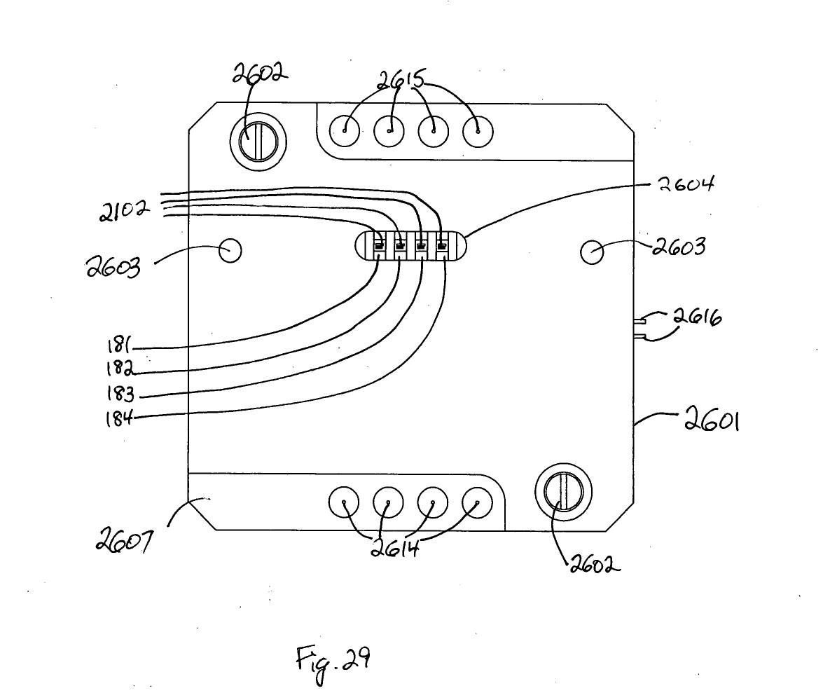

FIG. 26 is a drawing of perspective views of components of an exemplary microfluidics housing or cartridge which includes a heating element. FIG. 26A is a drawing of the cartridge lid, 2601, having a pair of screws for threadable attachment to the base block shown in FIG. 26D, and having alignment perforations, 2603, and a window perforation, 2604, for passage of electromagnetic beams for illuminating cantilevers within the cartridge. The alignment perforations or holes, 2603 are used for insertion of dowel pins, for alignment of the cartridge lid 2601 to the base, 2607 as shown in FIG. 29.

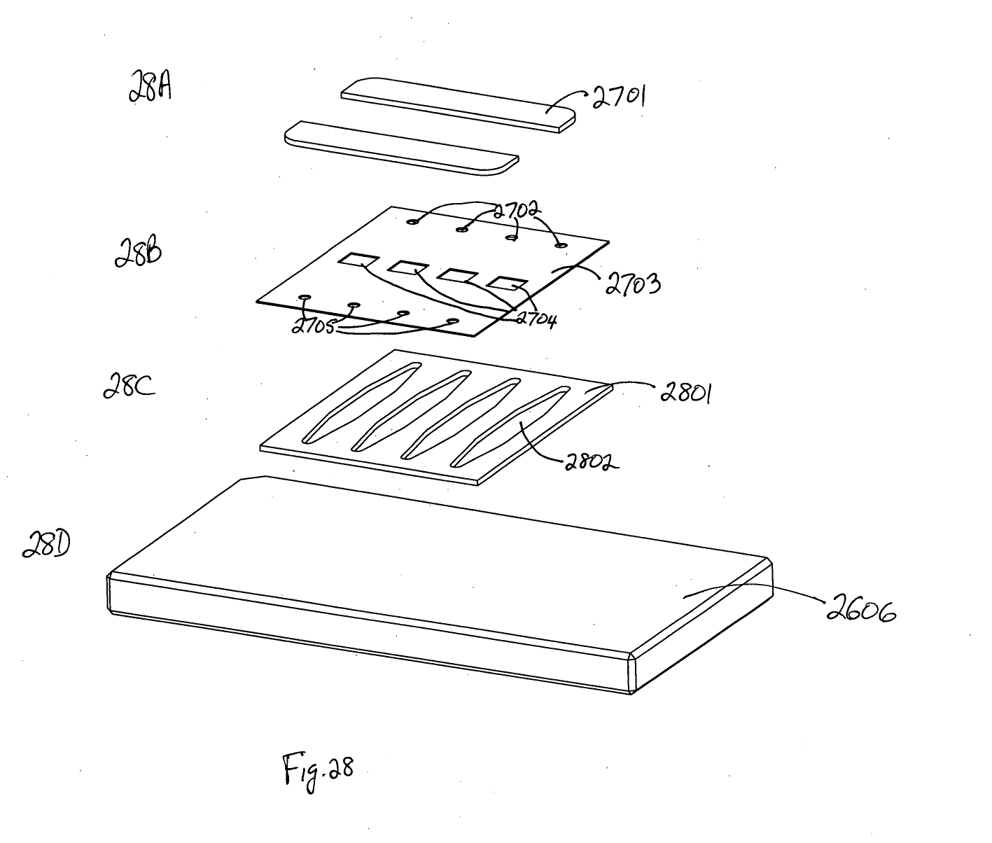

FIG. 26B is a drawing of the window substrate, 2606, which is manufactured of a transparent material, and from which are attached additional layers as shown in greater detail in FIG. 28 and identified here as 2605.

FIG. 26C is a drawing of the embodiment of the cantilever chip of FIGS. 21 and 22, 2101 indicating the arrangement of the chip as it is inserted in the embodiment of the cartridge.

FIG. 26D is a drawing of the cartridge base or block, 2607, which is manufactured from a metal block, milled to have features such a cutaway which is a pocket, 2608 for emplacement the cantilever chip, 2101. An additional layer 2609 located at each end of the pocket align the chip in position and secure that position during use.

A gasket, 2610 manufactured of silicone or similar flexible and resilient material is a separate part emplaced within the pocket prior to the chip, to lie under the cantilever chip and press against it to create a fluid seal, and serve as the floor or bottom of each of the plurality of the interaction cells that are formed by assembling the components. Also seen in the cartridge base are screw holes, 2612 to threadably receive the screws, 2602 seen in the lid, 2601 in FIG. 26A, when the entire cartridge is assembled, so that the interaction cells formed by assembly as described below are held tightly to form a fluid seal. Dowel pin holes, 2613 are visible on the top surface of the base block, and as shown as holes in the lid, 2603 serve to align the lid and the base. Inlet ports, 2614 and outlet ports 2615 for fluid connection using pumps are shown in the base, and are further illustrated in FIG. 29.

Wires for electrical connection, 2616 are shown. The cartridge functions both as a fluidics housing, and also for heating and heat maintenance for fluids delivered into the inlet ports, 2614 and entering into the interaction cells. The wires connect to the thermal control mechanism described below and shown in FIG. 29. Ideal thermal control warms incoming fluid and maintains a temperature within 0.1° C. of a desired temperature, however 0.2, or 0.5, or 1.0, 1.5 or 2.0° C. are also suitable levels of control in certain embodiments.

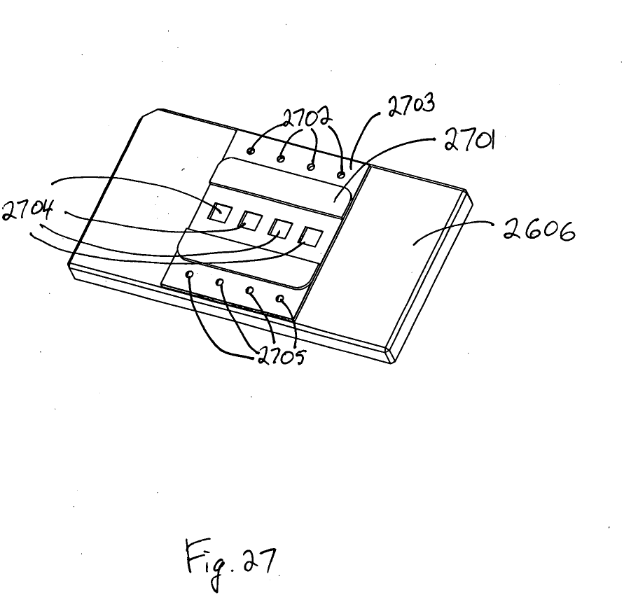

FIG. 27 is a drawing of a perspective view of the bottom of the window of FIG. 26B. The window is a piece of glass with channels for sample delivery, having four layers: a window substrate, a channel layer a via layer, and a support layer. When the assembled window is inverted and emplaced upon the cartridge base, 2607, it fits above the cantilevers of the cantilever chip, 2101 as shown in FIG. 26B. The via layer, 2703 is attached to the channel layer (not visible; see FIG. 28, 2801), and the channel layer is attached to the window substrate 2606 and includes a set of different perforations, including centrally arranged interaction cell perforations or holes, illustrated as squares or rectangles, 2704. The window substrate is a single piece of glass with optical coatings. Other layers can be manufactured of polydimethylsiloxane (PDMS) silicone, and are produced by fabricating PDMS sheets and cutting them into appropriate components using a laser. The layers are bonded to the glass and to each other using oxygen plasma activation.

Other perforations are outlet via holes, 2702, and inlet via holes, 2705. Exemplary dimensions for the interaction cell perforations are about 2.0 mm on the longitudinal side by about 2.15 mm on the transverse side, however many equivalent dimensions are within the scope of the conception. Exemplary dimensions for the inlet vial and outlet via perforations are about 1 mm in diameter. None of the dimensions herein or geometric shapes in the figures are intended to be construed as required for successful functioning of the apparatus, as other shapes and sizes are similarly functional.

FIG. 28 is a drawing of an exploded view of the layers of the window with perspective views of the layers of the window of FIGS. 26B and 27. FIG. 28A is a drawing of the two-part panels, 2701, of the support layer. FIG. 28B is a drawing of the via layer, 2703. FIG. 28C is a drawing of the channel layer, 2801 which has not been visible in figures above because it lies under the via layer and in contact with it. The channel layer contains four perforations for channels, 2802. The exact size and shape of these channels are exemplary and other sizes and shapes are within the conception of the device. Channels have an inlet section, a middle section which comprises the interaction cell or well, and an outlet section. Diameters of the channels may be, for example, 1.0 mm, 1.5 mm, 2.0 mm, up to 2.5 mm, however additional exemplary diameters for channels are within the scope of the microfluidics flow cell. The inlet and outlet sections are milled into the stainless steel of the cartridge and sealed with silicone. The middle section is fabricated in silicone and bonded to glass. FIG. 28D is a drawing of the transparent window substrate layer 2606 previously shown.

Fabrication of the channels is performed on the window substrate, comprising several layers of silicone. The multiple layers are stacked to produce the channels, and are bonded with plasma activation using widely known techniques. Channels are also milled into the stainless steel base block, and are sealed with single layers of silicone, which are held in place by aluminum plates.

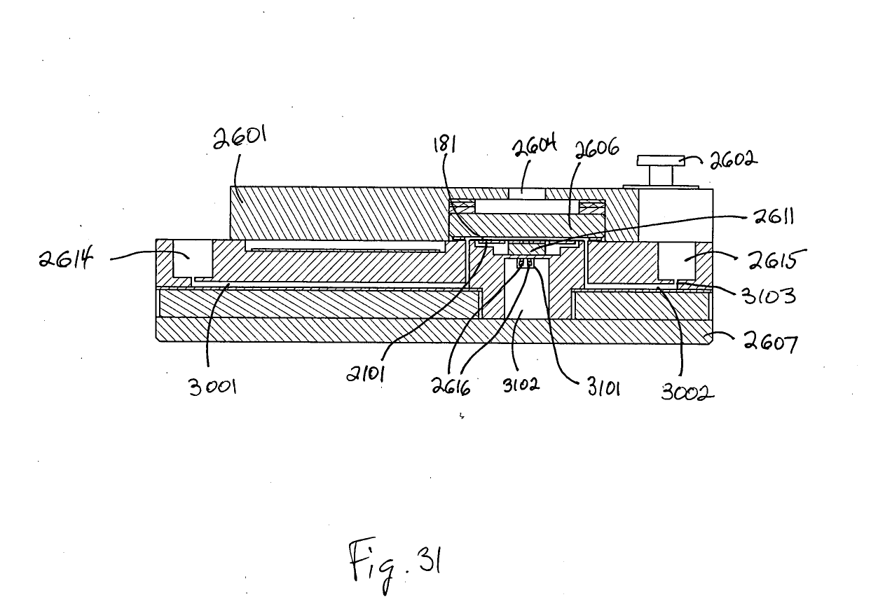

FIG. 29 is a drawing of a top view of the cartridge shown in FIG. 26A. The cartridge has a thermofoil heater and a temperature sensor, and functions both a heating unit for controllably heating incoming samples and buffers, and a housing for a flow cell for the microfluidics for the four wells or cells, 181-184 in which the cantilevers, 2102, visible through the window perforation, 2604 are exposed to various buffers and samples. The cartridge lid 2601 includes the previously illustrated screws, 2602 and alignment holes 2603. Also visible is the base block 2607 having inlet ports 2614 and outlet ports 2615 which are a series of four holes or perforations that are threaded and flat bottomed and as illustrated in this figure are about ¼ inch in depth. The inlet ports are connected to pumps for impelling fluid into the cartridge for sample analysis. The wires 2616 shown extending from the right hand side of the cartridge are electrical leads to supply electrical power to a temperature sensor and heating mechanism that are shown in FIG. 31.

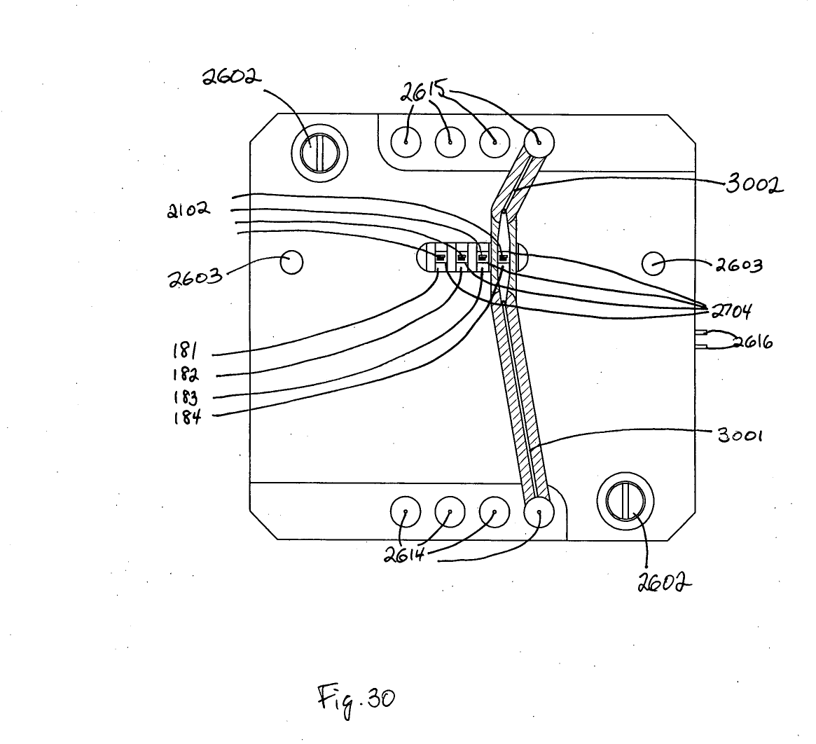

FIG. 30 is a drawing of a breakout view or longitudinal section of the cartridge as seen in a top view, showing, similarly to FIGS. 26 and 29 above, inlet ports, 2614 and outlet ports 2615 for fluid communication, screws, 2602, and alignment holes, 2603. Also visible are the cantilevers with reference plane, 2102, as seen through the perforations 2704 for illuminating cantilevers and analyzing reflection of beams for measurement of cantilever movement. An inlet channel, 3001, from the inlet port to the interaction cell is shown, through which a fluid material, such as a sample solution or a buffer, or any solution for cross-linking a ligand to the cantilever, is impelled, for example, using an external pump. An outlet channel, 3002, for further impelling fluid from the interaction cells, 181-184 for example, to either a waste receptacle or to a collection reservoir, is also shown. Fluid paths shown in more detail in FIG. 31 are milled into the base block.

FIG. 31 is a drawing of a view of the cartridge in cross section, showing the lid, 2601, assembled with the base block, 2607. The cross section view of the lid includes one of the screws, 2602, and a window perforation, 2604. The base block includes an a temperature sensor, 3101, a recess for the temperature sensor, 3102, and electrical leads, 2616 for the temperature control system. Further in the base are an outlet port, 2615, having a port exit, 3103, in fluid communication with a outlet channel 3002. The fluid path of the outlet channel is milled into the base block. A gasket, 2611, is located above the temperature sensor 3101, and provides the lower component of the interaction cell, 181-184, formed when the cartridge is assembled from the lid and base block. A cantilever chip 2101 is emplaced onto the surface of the gasket.

A fluid enters the cartridge from an inlet channel, 3105, via an inlet port, 2614, and is impelled into the interaction cell, 181. The fluid may be, for example, a sample fluid containing an analyte capable of interacting with a ligand on a cantilever, because the cantilever is configured to have the ligand on one surface, for the purpose of detecting the presence of the analyte by a deflection of the cantilever.

Although various exemplary embodiments of the invention have been disclosed, it should be apparent to those skilled in the art that various changes and modifications can be made which will achieve some of the advantages of the invention without departing from the true scope of the invention.

TABLE I
Example 1

| | | Total Vol μl | A | B | C | D |
|---|---|---|---|---|---|---|
| Step | Solution | (All Cells) | Reaction | Ref | NSB | BSA |
| 1 | DSU | 50 | + | + | 0 | + |
| 2 | Wash | 300 | + | + | + | + |
| 3 | Ab | 50 | + | + | + | + |
| 4 | Hbuffer | 50 | + | + | 0 | + |
| 5 | Wash | 300 | + | + | + | + |
| 6 | Sample | 50 | + | 0 | + | 0 |
| 7 | BSA | 50 | 0 | 0 | 0 | + |
| 8 | Wash | 300 | + | + | + | + |

Example 2

| | | Total Vol | A | | | |
|---|---|---|---|---|---|---|
| | | μl | Reac- | B | C | D |
| Step | Solution | (All Cells) | tion | Reaction | Reaction | Control |
| 1 | DSU | 50 | + | + | + | + |
| 2 | Wash | 300 | + | + | + | + |
| 3 | Ab | 50 | + | + | + | + |
| 4 | Hbuffer | 50 | + | + | + | + |
| 5 | Wash | 300 | + | + | + | + |
| 6 | Sample 1 | 50 | + | 0 | 0 | 0 |
| 7 | Sample 2 | 50 | 0 | + | 0 | 0 |
| 8 | Sample 3 | 50 | 0 | 0 | + | 0 |
| 9 | Wash | 300 | + | + | + | + |

Example 3

| | | Total Vol | | | | |
|---|---|---|---|---|---|---|
| | | μl | A | B | C | D |
| Step | Solution | (All Cells) | Reaction | Reaction | Reaction | Control |
| 1 | DSU | 50 | + | + | + | + |
| 2 | Wash | 300 | + | + | + | + |
| 3 | Ab 1 | 50 | + | 0 | 0 | 0 |
| 4 | Ab 2 | 50 | 0 | + | 0 | 0 |
| 5 | Ab 3 | 50 | 0 | 0 | + | 0 |
| 6 | Hbuffer | 50 | + | + | + | + |
| 7 | Wash | 300 | + | + | + | + |
| 8 | Sample | 50 | + | + | + | 0 |
| 9 | Wash | 300 | + | + | + | + |

What is claimed is:

1. A cantilever sensor device comprising:
a cantilever die comprising a plurality of cantilevers, each cantilever having a base end attached to a base and a free end that deflects as a response to a presence of an analyte in a sample in an environment, the base further comprising a reference plane as a control for non-analyte signals in the environment.

2. The device according to claim 1, the reference plane is co-contiguous to the base, and the base, reference plain and cantilevers are substantially co-planar or occupy parallel planes.

3. The device according to claim 1, wherein the cantilevers, the base and the reference plane comprise a die having layers of substantially the same substrate materials.

4. The device according to claim 3, wherein the substrate further includes a coating comprising a ligand for the analyte.

5. The device according to claim 3, wherein the substrate further includes a coating absent a ligand for the analyte.

6. The device according to claim 3, wherein a first side of the cantilevers, the base and the reference plane each comprises the coating.

7. The device according to claim 1, wherein the reference plane measures non-analyte signals comprising at least one from the group: thermal variation within the opto-mechanical assembly; refractive index difference in sample fluids; and drift in electronics.

8. The device according to claim 7, wherein drift in electronics is a variation in at least one of: in intensity of a laser source, movement of a laser source, sensitivity of a position sensitive detector (PSD, and sensitivity of a charge couple device (CCD).

9. The device according to claim 1 further comprising an algorithm embedded in a computer-readable medium, wherein the algorithm integrates a sum of non-analyte signal data and subtracts the sum from an amount of cantilever responses.

10. The device according to claim 9, wherein the cantilever responses comprise at least one change of: extent of deflection; resonance frequency; higher flexural mode; phase angle of the flexural mode with respect to an actuation mechanism; and a quality factor of the flexural modes.

11. A cantilever platform comprising:
a plurality of interaction cells, each interaction cell comprising an inlet for receiving a sample fluid; and
at least one test cantilever having a movable end and a fixed base disposed in each interaction cell, and at least one reference plane fixed relative to the cantilever base, wherein the test cantilever and the reference plane comprise a die having layers of substantially the same substrate materials.

12. The cantilever platform according to claim 11, wherein each interaction cell further includes at least one outlet whereby fluid may flow out of the cell.

13. The cantilever platform according to claim 12, further comprising a housing.

14. The cantilever platform according to claim 13, wherein the housing is a fluidics cartridge comprising the plurality of interaction cells, inlets and outlets, and a lid.

15. The cantilever platform according to claim 10, wherein the die comprising the test cantilevers and reference plane disposed in the interaction cell is removable.

16. The cantilever platform according to claim 11, further comprising a plurality of test cantilevers.

17. The cantilever platform according to claim 16, wherein the plurality of test cantilevers is contiguous to the reference plane.

18. The cantilever platform according to claim 14, wherein the fluidics cartridge comprises a top and a bottom, the bottom comprising a thermal sensor and electrical connection for thermal control, and the fluid lines entering the cells are temperature controlled.

19. The cantilever platform according to claim 14, wherein the housing is removably inserted into an illuminator-reader apparatus for measuring a movement of a test cantilever and an apparent movement of the reference plane.

20. The cantilever platform according to claim 19, wherein the illuminator-reader apparatus further comprises means for generating and focusing a plurality of laser beams, wherein at least one of the plurality of laser beams focuses respectively on each of the test cantilevers and on the reference plane.

21. A method for analyzing in each of a plurality of sample fluids a quantity of an analyte or a change in conformation of a ligand in which non-analyte factors are detected, the method comprising:
contacting at least one of a plurality of interaction cells with at least one of the sample fluids, wherein each of the interaction cells comprises at least one test cantilever and at least one reference plane;
detecting in each interaction cell a response of the test cantilever and an amount of an apparent deflection of the reference plane; and
calculating a difference in the response of the test cantilever and the amount of the apparent deflection of the reference plane cantilever due to non-analyte factors, wherein the quantity of the analyte or change in conformation of the ligand is calculated.

22. A method according to claim 21, wherein the test cantilever is configured to deflect in response to the presence of a ligand selected from a group consisting of a protein and a nucleic acid.

23. A method according to claim 22, wherein the nucleic acid is RNA.

24. A method according to claim 22, wherein the nucleic acid is DNA.

25. A method according to claim 22, wherein the protein is selected from an epitope, an enzyme, and a synthetic polypeptide.

26. A method according to claim 22, wherein the ligand or the analyte is a substrate for an enzyme.

27. A method according to claim 22, wherein the ligand or the analyte is a hormone.

28. A method according to claim 25, wherein the hormone is selected from the group consisting of a steroid and a polypeptide.

29. A method according to claim 22, wherein the ligand or analyte is selected from the group consisting of an antibody and an antigen.

30. A method according to claim 22, further comprising mounting a die comprising the at least one cantilever for the plurality of interaction cells in a housing comprising a bottom portion having means for controlling temperature of the cells and means for controlling temperature of the fluid entering the cells.

31. A method according to claim 22, wherein detecting an amount of an apparent deflection is measuring a sum of non-analyte signals.

32. The method according to claim 22, wherein the non-analyte factors comprise at least one of: thermal variation within the opto-mechanical assembly; refractive index difference in sample fluids; and drift in electronics.

33. A method according to claim 22, wherein calculating the difference further comprises using a computer algorithm embedded in a computer readable medium.

34. A microfluidics device comprising:
four interaction cells, each interaction cells being configured to contain at least four test cantilevers and one reference plane; and
fluid control means for providing to the interaction cells a sample fluid, wherein each interaction cell receives a different sample fluid.

35. The microfluidics device according to claim 34, wherein the device comprises a bottom portion and a top bottom, wherein emplacement of the top upon the bottom forms the interaction cells and the means wherein each cell receives a different fluid.

36. The microfluidics device according to claim 35, wherein the bottom portion comprises thermal control means.

37. The microfluidics device according to claim 36, wherein thermal control means comprises a sensor, a heater, and electrical connections.
